(19) United States
(12) Patent Application Publication
(10) Pub. No.: US 2023/0143359 A1
(43) Pub. Date: May 11, 2023

(54) GENE INTERFERENCE VECTOR- AND IRON NANOPARTICLE-BASED COMPOSITION FOR KILLING CANCER CELLS, AND USE THEREOF

(71) Applicant: SOUTHEAST UNIVERSITY, Nanjing (CN)

(72) Inventors: Jinke WANG, Nanjing (CN); Jinliang Gao, Nanjing (CN); Tao Luo, Nanjing (CN)

(73) Assignee: SOUTHEAST UNIVERSITY, Nanjing (CN)

(21) Appl. No.: 17/911,130

(22) PCT Filed: Jan. 15, 2021
(86) PCT No.: PCT/CN2021/072025

§ 371 Date: Sep. 12, 2022

(30) Foreign Application Priority Data
Mar. 12, 2020 (CN) 202010173472.2

(51) Int. Cl. A61K 33/26; A61K 48/00; A61P 35/00

(52) U.S. Cl. CPC A61K 33/26; A61K 48/0058; A61P 35/00

(57) ABSTRACT

Disclosed in the present invention are a gene interference vector- and iron nanoparticle-based composition for killing cancer cells, and the use thereof. The composition comprises a gene interference vector and iron nanoparticles, wherein the gene interference vector is a CRISPR/Cas13a expression vector or microRNA expression vector controlled by a cancer cell specific promoter DMP, with the Cas13a-gRNA or microRNA expressed by the vector being able to inhibit, in a targeted manner, intracellular iron metabolism and the expression of reactive oxygen related genes, and the iron nanoparticles can be degraded after entering cells to produce iron ions and to increase the reactive oxygen level. The composition comprising the gene interference vector and the iron nanoparticles of the present invention can be used for preparing a new reagent for treating cancers.

### TECHNICAL FIELD

The invention relates to the field of cancer gene therapy biotechnology, in particular to a composition for killing cancer cells based on a gene interference vector and iron nanoparticles and its application.

### BACKGROUND TECHNOLOGY

Cancer is an important disease that troubles human health and threatens human life. In the long-term battle against cancer, human beings have developed a variety of cancer treatment methods such as surgery, chemotherapy, targeted therapy, and immunotherapy, and have also made significant progress, and the cancer survival rate has been significantly improved. However, the current level of cancer treatment is still far from the ideal for the majority of patients in terms of health and life. Therefore, actively developing new cancer treatment technologies is still the goal of continuous efforts in the medical field. Gene therapy is a frontier field and a technological highland of future medicine. The progress made by gene therapy in the field of genetic disease treatment has attracted the attention of the medical community, but there has been no significant breakthrough in the use of this technology in cancer treatment. Therefore, the exploration of cancer single gene therapy also advances progress and breakthrough in the development of new cancer treatment technologies.

Cancer gene therapy is to introduce genetic material into cancer cells, and use the genetic material to achieve a therapeutic result, interfere with the growth of cancer cells or kill cancer cells. In particular, the introduction of a certain gene into cells can inhibit cancer cell growth, or causing cancer cells apoptosis and necrosis, by expressing gene products such as interfering RNA or protein. Two critical issues in gene therapy, are 1) the selection of genes, which directly determines the efficiency of treatment; and 2) to control the expression of genes only in cancer cells, that is, cancer cell specificity. Relatively speaking, the selection of genes is not very challenging. Based on the current genomic science research on a large number of gene functions, many genes, especially genes encoding interfering RNAs and their tumor suppressor proteins, will more or less, after being introduced into cells, inhibit the growth on cancer cells. The more critical issue is how to control the gene expression only in cancer cells, that is, cancer cell specificity. At present, based on the principles of synthetic biology, some gene switches have been developed to control the specific expression of genes in cancer cells, but these gene control elements are complex in composition, low in efficiency, and still far from application.

As a fundamental biological phenomenon of cells, programmed cell death (PCD) plays an important role in eliminating unwanted or abnormal cells in multicellular organisms, which is essential for normal development, homeostasis, and the prevention of hyperproliferative diseases (such as cancer) is crucial. Recently, as a new type of PCD, ferroptosis has attracted more and more attention. In 2012, Stockwell et al. identified ferroptosis as an iron-dependent form of non-apoptotic regulated cell death. Ferroptosis is dependent on intracellular iron, independent of other metals, and is morphologically, biochemically and genetically not related to other well-known regulated cell death types such as apoptosis, necrosis, necroptosis and autophagy. However, ferroptosis is associated with elevated levels of intracellular reactive oxygen species (ROS), which can be prevented by iron chelation or genetic inhibition of cellular iron uptake. Inactivation of cellular components by glutathione peroxidase 4 (GPX4) induces an iron-dependent form of cell death, as this leads to the accumulation of ROS on membrane lipids.

Although ROS have been shown to regulate cell survival, high levels of ROS can cause irreversible cellular damage, leading to apoptosis, autophagy, and necrosis in various types of cancer cells. So far, many studies have demonstrated that certain natural products can generate specific anti-cancerous effects on breast cancer cancel cells by up-regulating ROS levels, suggesting that ROS may mediate selective activation of apoptosis to specifically kill cancer cells. When ferrous iron (Fe²⁺) exists together with peroxides and oxygen, ROS can be generated through the Fenton reaction. Iron is not only directly involved in many reactions related to ferroptosis, but is also responsible for the accumulation of ROS mediated by the Fenton reaction, as demonstrated by increased iron uptake and inhibition by iron chelators. ROS levels are usually balanced by the combination of antioxidant production and iron transport systems, typically including transferrin uptake, ferritin storage, and ferroportin (FPN). Three proteins, including transferrin (Tf), transferrin receptor 1 (TFR1) and FPN, play key roles in regulating the balance of iron content in the body.

Tumors have a particularly high demand for iron compared to normal cells due to the unique physiological processes of cancer cells. Using opportunistic nutritional acquisition is considered as one of the hallmarks of cancer cells. Numerous studies have shown that cancer cells tend to upregulate TFR1 expression to increase iron uptake and downregulate FPN expression to reduce iron efflux and increase iron retention. However, under iron overload conditions, cancer cells are more likely to accumulate ROS than normal cells, thereby exacerbating ferroptosis. Therefore, the regulation of iron may provide new therapeutic opportunities for cancer. To date, FPN is the only known cellular exporter of iron in mammals. Recently, FPN has been found to be dysregulated in many cancers, such as breast, prostate, ovarian, colorectal, and multiple myeloma, and leukemia cell lines have also been associated with low FPN expression relative to normal bone marrow.

Although iron absorption, storage, and excretion are well regulated, administration of iron in nanoparticle form provides an unnatural route for iron to enter cells. Many studies have reported that iron-based nanomaterials can accumulate at tumor sites due to their ability to passively and actively target, and that iron released as ferrous (Fe²⁺) or iron (Fe³⁺) ions in acidic lysosomes participates in the Fenton reaction and induces ferroptosis to kill cancer cells. However, due to the importance of iron to cells, cells have evolved a set of mechanisms and systems to maintain intracellular iron balance (iron homeostasis); under the action of this system, cells can effectively export excess iron ions in cells; therefore although a large number of literatures have reported that iron-based nanoparticles have the effect of causing ferroptosis, due to the role of the cellular iron homeostasis system, the iron ions released by iron-based nanoparticles in cells will soon be exported out of cells, so simply using iron-based nanoparticles have very limited effect on inhibiting the growth of cancer cells by utilizing the iron apoptosis mechanism, and have no clinical development value.

### SUMMARY OF THE INVENTION

Object of the invention: In view of the existing problems of cancer gene therapy and iron nanomaterials inducing ferroptosis of cancer cells, the present invention discloses a composition for killing cancer cells and its application. The present invention specifically discloses34 a composition of gene interference vector and iron nanoparticles for killing cancer cells, the present invention also provides a new method for killing cancer cells using the same. The new method comprises using gene interference vector and iron nanoparticle. The particle is a combination of two biological and chemical materials to kill cancer cells.

Technical solution: in order to achieve the above purpose, a composition for killing cancer cells according to the present invention is characterized in that it comprises a gene interference vector and iron nanoparticles, and the gene interference vector is a cancer cell specific promoter DMP-controlled CRISPR/Cas13a expression vector or microRNA expression vector.

Wherein, the Cas13a-gRNA expressed by the CRISPR/Cas13a expression vector, or the microRNA expressed by the microRNA expression vector can target and inhibit the expression of target genes in cells, specifically, can target and inhibit intracellular iron metabolism and reactive oxygen species-related gene expression.

The iron nanoparticles are iron nanomaterials that can be degraded to generate iron ions after entering cells and lead to an increase in a level of intracellular reactive oxygen species.

Preferably, the iron nanomaterials are ferric oxide nanoparticles (Fe₃O₄@DMSA) modified by Dimethylaminosulfanilide (DMSA) (FeNPs for short).

Further, the cancer cell-specific promoter DMP promoter is a NF-κB specific promoter, which is formed by linking an NF-κB decoy and a minimal promoter (patent application number CN201710812983.2), and the DMP promoter can activate its downstream genes to be expressed in various cancer cells except in normal cells (patent application numbers CN201711335257.2, CN201810163823.4); the DMP promoter can control the expression of CRISPR/Cas13a or microRNA expression vector in cancer cells specific expression.

Wherein, in the CRISPR/Cas13a expression vector, the expression of Cas13a is controlled by the DMP promoter, and the expression of gRNA is controlled by the U6 promoter (Chinese Patent Application No. 202010096220.4). The functional DNA elements and sequences of the CRISPR/Cas13a expression vector are shown in FIG. 1. The expression of microRNA is controlled by the DMP promoter in the microRNA expression vector (Chinese patent application number 201710812983.2); the functional DNA elements and sequence of the microRNA expression vector are shown in FIG. 2 (usually microRNA can be abbreviated as miRNA).

Preferably, the DNA sequence of the functional element of the CRISPR/Cas13a expression vector (pDMP-Cas13a-U6-gRNA; pDCUg for abbreviation) is shown in SEQ ID NO.1; the microRNA expression vector (pDMP-miR) The DNA sequence of the functional element is shown in SEQ ID NO.2.

Further, the CRISPR/Cas13a or microRNA expression vector can express either gRNA or microRNA targeting a single gene, or can co-express gRNA or microRNA targeting multiple genes.

Preferably, the iron metabolism and reactive oxygen species related genes mainly include FPN, LCN2, FSP1, FTH1, GPX4, NRF2 and SLC7A11 genes.

Further, the CRISPR/Cas13a or microRNA expression vector can express gRNA or microRNA targeting FPN, LCN2, FSP1, FTH1, GPX4, NRF2 and SLC7A11 genes; the gRNA can form a first complex with Cas13a protein, and the microRNA can interact with RISC forming a second complex, and both the first and second complexes can target cleavage of the mRNA of the above-mentioned genes, resulting in a decrease in the expression level of the proteins encoded by the above-mentioned genes.

Preferably, the target binding sequences of the gRNA targeting FPN and LCN2 are:

(FPN)
5′-CACCG CAAAG TGCCA CATCC GATCT CCC-3′
and
(LCN2)
5′-TAACT CTTAATGTTG CCCAG CGTGA ACT-3′;

the target binding sequences of the microRNAs targeting FPN, LCN2, FSP1, FTH1, GPX4, NRF2 and SLC7A11 genes are:

(FPN)
5′-TCTAC CTGCA GCTTA CATGAT-3′,
(LCN2)
5′-TAATG TTGCC CAGCG TGAAC T-3′,
(FSP1)
5′-CAAAC AAACA AATAA AGTGG A-3′,
(FSP1)
5′-TAAAC AAACA AACAA ATAAA G-3′,
(FTH1)
5′-ATCCC AAGAC CTCAA AGACA A-3′,
(FTH1)
5′-TAAGG AATCT GGAAG ATAGC C-3′,
(GPX4)
5′-TTCAG TAGGC GGCAA AGGCG G-3′,
(GPX4)
AGGAA CTGTG GAGAG ACGGT G-3′,
(NRF2)
5′-TACTG ATTCA ACATA CTGAC A-3′,
(NRF2)
5′-TTTAC ACTTA CACAG AAACT A-3′,
(SLC7A11)
5′-AAATG ATACA GCCTT AACAC A-3′,
and
(SLC7A11)
5′-TTGAG TTGAG GACCA GTTAG T-3′.

Preferably, the iron nanoparticles or iron nanomaterials are DMSA-modified Fe₃O₄ nanoparticles (FeNPs) or PEI-modified Fe₃O₄ nanoparticles (FeNCs). The two iron nanomaterials can be prepared or obtained commercially.

Furthermore, under the combined action of gene interference vector and iron nanoparticles, the levels of iron ions and reactive oxygen species in cancer cells can be sharply increased, which can induce significant iron apoptosis in cancer cells.

Wherein, the gene interference vector can be administered in vivo in the form of a viral vector or a non-viral vector; the iron nanoparticles can be administered in vivo as a separate chemical material, or can be simultaneously administered as a nanocarrier of a gene interference vector in vivo.

Preferably, the viral vector is an adeno-associated virus (AAV), and the non-viral vector is a nanocarrier.

Further, nanocarriers are iron nanoparticles that can bind DNA.

Preferably, the DNA-binding iron nanoparticles are polyethylenimine (PEI) modified iron tetroxide nanoparticles (Fe₃O₄@PEI) (FeNCs for abbreviation).

The application of the composition for killing cancer cells of the present invention in the preparation of novel cancer treatment reagents. Specifically, it refers to the application of the combination of two biological and chemical materials, gene interference vectors and iron nanoparticles, in the preparation of novel cancer treatment reagents.

Specifically, the reagent includes two components: a gene interference vector and iron nanoparticles; the gene interference vector includes a DMP-controlled CRISPR/Cas13a or microRNA expression vector; the gene interference vector can be either plasmid DNA or linear DNA; wherein iron nanoparticles include various iron nanoparticles, preferably, iron nanoparticles are FeNPs and FeNCs; wherein FeNCs have dual functions, being both iron nanoparticles and a nanocarrier for gene interference vectors.

In the present invention, a composition for killing cancer cells is developed, which includes a gene interference vector and iron nanoparticles and a new method for killing cancer cells based on the gene interference vector and iron nanoparticles. The invention combines the iron-based nanomaterials with the gene expression regulation technology controlled by the NF-κB specific promoter DMP. Controlled by the DMP promoter are two gene expression interference tools, CRISPR/Cas13a and microRNA, which inhibit the expression of iron metabolism and reactive oxygen species (ROS)-related genes in cancer cells, and cooperate with iron nanoparticles to degrade after entering cells to form iron ions and generate free radicals, resulting in a sharp increase in the levels of intracellular iron ions and ROS, resulting in significant ferroptosis in cancer cells. In the present invention, the expression of two iron metabolism-related genes FPN and Lcn2 in three leukemia cells KG-1a, HL60 and WEHI-3 is firstly inhibited by the DMP-controlled CRISPR/Cas13a and microRNA expression vector, and iron nanoparticles are combined Significantly increased the level of ROS in leukemia cells, causing significant ferroptosis in leukemia cells. Multiple cancer cell lines representing 10 common solid tumors were then treated in the same way with similar results. Explain that the composition of the present invention and the treatment method thereof not only have an anti-cancerous effect on blood cancer cells, but also have a anti-cancerous effect on various solid tumor cancer cells. Therefore, the composition of the present invention and the method for killing cancer cells are: A new technology for broad-spectrum cancer cell killing. In addition, the DMP-controlled CRISPR/Cas13a and microRNA expression vectors targeting FPN and Lcn2 genes were packaged into AAV viruses, which were combined with the same intravenous injection of iron nanoparticles to significantly inhibit the proliferation of leukemia cells in mice. It shows that the proliferation of cancer cells can be inhibited both in vitro and in vivo. Therefore, the composition based on the gene interference vector and iron nanoparticles for killing cancer cells proposed by the present invention, the combination of the two biological and chemical materials, the gene interference vector and the iron nanoparticles, has potential application value in the preparation of novel cancer therapeutic agents.

Beneficial effect: compared with the prior arts, the present invention has the following advantages.

1. Compared with the existing causing cancer death technology that has been clinically applied, the present invention discloses a new anti-cancer cell composition in principle, that is, a composition for Gene Interfered Forroptosis Therapy (GIFT) enhanced by gene interference, in another words, a composition based on a gene interference vector and iron nanoparticles for killing cancer cells.

Iron-based nanoparticles have been successfully used as MRI imaging in clinical diagnosis of cancer and clinical treatment of anemia, but iron-based nanoparticles have not been used in clinical treatment of cancer based on their chemical nature. However, a large number of studies have shown that iron-based nanoparticles are degraded in the acidic environment of intracellular lysosomes to release iron ions, which in turn leads to increased intracellular ROS levels and induce apoptosis. This process coincides with the mechanism of ferroptosis that has been extensively studied and revealed in recent years. However, due to the importance of iron to cells, cells have evolved a set of mechanisms and systems to maintain intracellular iron balance (iron homeostasis); under the balance action of this system, cells can effectively export excess iron ions out of the cells; therefore although a large number of literatures have reported that iron-based nanoparticles have the effect of causing ferroptosis, due to the effect of the cellular iron homeostasis system, the iron ions released by iron-based nanoparticles in cells will soon be exported to cells, so simply using iron-based nanoparticles have very limited effects on inhibiting the growth of cancer cells by utilizing the iron apoptosis mechanism, and have little clinical value.

The inventors of the present invention found that when cells were treated with DMSA-modified Fe₃O₄ nanoparticles (FeNPs), FeNPs would enter cells and degrade in the acidic environment of lysosomes, releasing iron ions, resulting in an increase in the level of intracellular iron ions; in order to maintain iron at a steady state, cells respond to increased iron efflux-related gene expression, most notably FPN and Lcn2.

In the present invention, FPN and Lcn2 are used as important targets for killing cancer cells by the mechanism of iron apoptosis. It is believed that in the case of knocking down the expression of these two iron export-related genes, treating cells with iron nanomaterials will cause an increase in the level of intracellular iron ions; since the generated iron ions cannot be effectively exported out of the cells, it will cause a massive accumulation of internal iron ions and the sharp increase of ROS, thus induce significant ferroptosis in cells, but this mechanism works for both normal cells and cancer cells. Therefore, the most critical question is how to control to suppress (or knock down) the expression of these two iron export-related genes only in cancer cells without disturbing the expression of these two genes in normal cells.

In the applicant's past research, a cancer cell-specific promoter, the DMP promoter, was designed and demonstrated, which was formed by linking the NF-κB decoy (Decoy) sequence with the Minimal Promoter (Int. J. Biochem. Cell. Biol. 2018, 95:43-52; Patent Application No. CN201710812983.2), and proved that this promoter can drive its downstream expression in various cancer cells, but not in normal cells (Hum Gene Ther. 2019, 30:471-484; Gene Therapy 2020, DOI: https://doi.org/10.1038/s41434-020-0128-x; patent application numbers CN201711335257.2, CN201810163823.4). Therefore, the present invention uses DMP to control the expression of gene interference tools such as CRISPR/Cas13 and miRNA in cells to specifically knock down the expression of iron export-related genes FPN and Lcn2 in cancer cells without affecting their expression in normal cells.

Based on the above reasoning, in the present invention, a cancer cell killing composition based on a gene interference vector and iron nanoparticles is proposed. The composition combines iron-based nanomaterials with a gene expression interference tool controlled by a cancer cell-specific promoter DMP. In the present invention, the DMP promoter is used to control the intracellular expression of two gene interference tools, CRISPR/Cas13 and miRNA, and a gene knockdown vector targeting FPN and Lcn2 mRNA is constructed. Using these vectors in combination with a type of iron nanoparticles (FeNPs), the effect of this combination on various cancer cells as well as normal cells was observed. The results showed that this combination had a significant anticancerous effect on various cancer cells, while it had no effect on normal cells. And it is proved that neither of the two alone can produce significant anticancerous effect on cancer cells, nor does it have significant effect on normal cells. By packaging the gene knockdown vector into AAV virus and injecting the resulting recombinant virus with FeNPs into mice intravenously, it was found that the combination of the two can significantly inhibit the growth of subcutaneous xenografts in mice. These results fully demonstrate the feasibility and reliability of the compositions and method thereof. In addition, by measuring the intracellular iron content, ROS level, and the expression of the effector gene Cas13 and two target genes in vitro and in vivo, it was further demonstrated that the mechanism of the composition and method for killing cancer cells is the genetic interference enhanced iron Forroptosis Therapy (GIFT) designed by the present invention.

2. The novel composition and its novel method for killing cancer cells proposed by the present invention have three significant advantages, namely cancer cell specificity, significant efficacy and applicable to a broad spectrum of cancer cells.

In the present invention, the expression system of CRISPR/Cas13a and miRNA controlled by DMP is firstly targeted to inhibit the expression of two iron metabolism-related genes FPN and Lcn2 in three leukemia cells KG-1a, HL60 and WEHI-3. Together with iron nanoparticles, it is found the composition significantly increased the levels of ROS in leukemia cells, causing significant ferroptosis in leukemia cells. Multiple cancer cell lines representing 10 common solid tumors (14 in total) were subsequently treated with the same approach, yielding similar results. These experiments demonstrate that the composition and the method for killing cancer cells not only have works on blood cancer cells, but also have can cause death on various solid tumor cancer cells. Therefore, the composition and the method for cancer cell treatment are a novel broad-spectrum anti-cancer cell technology. Experimental studies have shown that all kinds of cancer cells are basically killed in 72 hours after the composition of the present invention and the treatment are administered, and the death rate of the cancer cells is extremely significant. By treating three normal cells (human normal hepatocytes HL7702, human embryonic fibroblasts MRC5 and human gastric mucosa epithelial cells GES-1), it was demonstrated that the new composition and its anti-cancer treatment method have no effect on the growth of normal cells. This experiment and its significant impact demonstrate the cancer cell specificity of the new composition and its anti-cancer treatment method. In addition, in vivo experiments also showed that Cas13a and microRNA controlled by DMP are only expressed in tumor tissues, but not in normal tissues, indicating that the anti-cancer cell treatment including the composition and the method for proposed by the present invention also has cancer cell specificity in vivo, that is, it only works in tumor tissue.

3. The composition and the new anti-cancer treatment method proposed by the present invention is flexible and feasible in terms of administration means and dosage forms for anti-cancer treatment in vivo.

To demonstrate the possibility and feasibility of in vivo administration for anti-cancer cells treatment in vivo, the present invention has carried out three batches of animal experiments, respectively trying to deliver gene interference vector DNA in vivo by using viral vectors and iron nanomaterials as non-viral vectors. In the first batch of animal experiments, rAAV virus and FeNPs were injected intravenously twice, and FeNPs were injected the next day after rAAV injection; in the second batch of animal experiments, in order to further simplify the administration method, rAAV and FeNPs were injected. After mixing in vitro, it is administered by one-time intravenous injection. The results showed that the two sequential administrations of rAAV and FeNPs and the one-time simultaneous administration of the two generate similar antitumor effects, which provides a more convenient pathway for the in vivo application of the composition disclosed herein and its method for killing cancer cells.

Traditionally, DNA delivery systems are divided into viral vector-mediated systems and non-viral vector-mediated systems, where non-viral pathways have become powerful and popular research tools for elucidating gene structure, regulation, and function. Virus-mediated gene delivery systems are currently the main gene delivery systems for in vivo gene therapy due to their high efficiency. For example, several gene therapies approved by the FDA for clinical treatment, as well as a large number of clinical studies, all use AAV as a gene delivery tool. However, the most critical drawbacks of virus-mediated gene delivery systems are the potential immune response and the long cycle and high cost of virus preparation; in addition, since AAV virus is a naturally occurring virus in the human body, it is present in many individuals, along with natural antibodies and immune memory against it. The wide application of AAV virus in all individuals is very limited. Consequently, a large number of non-viral vector systems have been researched and developed over the past decade. These include Magnetofection™, a magnetic nanotransfection agent for nucleic acid delivery that has been developed (Ther Deliv. 2011; 2:717-26).

In order to overcome the shortcomings of AAV vectors and make full use of the chemical properties of magnetic nanotransfection materials, the present invention also attempts to use iron nanoparticles as plasmid DNA nanocarriers (nanocarriers), called Fe nanocarriers (FeNCs). Cancer cell inhibition assays to further simplify reagent preparation and reduce costs. The FeNCs used in the present invention are Fe₃O₄ nanoparticles modified by polyethyleneimine (Polyethylenimine, PEI). In the present invention, the magnetic transfection agent can not only be used as a carrier for DNA delivery in vivo, but also can be used as an iron nano-donor. The results of the third batch of animal experiments showed that FeNCs loaded with plasmid DNA (abbreviated as FeNCs@DNA) could also significantly knock down the expression of FPN and Lcn2 genes in tumor tissue in mice by intravenous injection, and significantly inhibit tumor growth. Therefore, the present invention also develops a new dosage form for inhibiting the growth of cancer cells by a new method. The dosage form and its two components (plasmid DNA and FeNCs) can not only be commercially manufactured in vitro on a large scale, but also with a short production cycle at low cost, these characteristic making it a very promising candidate for the development of novel cancer therapeutics. In addition, the dosage form avoids possible immune responses from the use of the virus and is expected to be applicable to all individuals.

4. The gene interference vector proposed by the present invention is very beneficial to the in vivo application of the composition and the anti-cancer treatment method of the present invention.

In the present invention, DMP is used to control two gene interference systems CRISPR/Cas13-gRNA and miRNA to achieve the purpose of inhibiting the expression of target genes in cancer cells in vitro and in vivo, and the cooperation of DMP and the two gene interference systems is very beneficial to in vivo application of the new composition and its anti-cancer treatment methods and create simultaneous multigene interference.

In the present invention, the most commonly used and safest adeno-associated virus (AAV) in gene therapy is used as the carrier for gene interference vector (vector) in vivo delivery, but the disadvantage of AVV is that its DNA packaging capacity is limited, and generally cannot package more than 4 Kb DNA fragments. The DMP promoter and the two gene interference systems CRISPR/Cas13-gRNA and miRNA used in the present invention are very advantageous in the application of AAV for in vivo delivery and multigene co-suppression (or knockdown). For example, the present invention co-expresses FPN and Lcn2, and co-expresses other 5 target genes (miFFGNS). Since the DMP promoter is very short (84 bp), and Cas13 can process its own gRNA precursor, when constructing gRNA targeting multiple genes, only one U6 promoter is needed to direct the transcription of one precursor RNA, and this precursor RNA can be processed by Cas13 to form mature gRNA that can target multiple genes or targets, respectively, such as pDCUg-hFL or pDCUg-mFL in the present invention. Being short, an advantage for this DMP promoter and Cas13a-gRNA, is very beneficial to package the Cas13 expression vector (DCUg) sequence that can target multiple genes or targets into an AVV particle, such as the rAAV-DCUg-hFL or rAAV-pDCUg-mFL. The pDMP-miRNA vector used in the present invention is also very advantageous in making a vector targeting multiple genes or multiple targets. In the pDMP-miR vector used in the present invention, the DMP promoter is only 84 bp, each miRNA backbone is only 341 bp, the HSV TK poly(A) signal is only 49 bp, and a complete DMP-miRNA expression unit is only 474 bp in total, which is very beneficial to tandem combination of DMP-miRNA units targeting multiple genes or multiple targets to construct co-expression pDMP-miRNA vectors targeting multiple genes or multiple targets, such as pDMhFL or pDMmFL. This polygenic co-suppression has important implications. The present invention finds that the co-expression of gRNA or miRNA targeting multiple iron metabolism or ROS regulation related genes (such as FPN and Lcn2) has a significant synergistic effect in killing cancer cells, and can be compatible with FeNPs to produce the greatest cancer cell anticancerous effect (eg pDCUg-hFL or pDCUg-mFL, pDMhFL/pDMmFL).

5. The new composition and anti-cancer treatment method for killing cancer cells proposed by the present invention are expected to solve the drug resistance problem of cancer cells.

Chemotherapy is one of the main therapies for cancer treatment at present, but chemoresistance is still a huge obstacle to cancer cure. Therefore, there is an urgent need to seek new treatment strategies for people who no longer benefit from chemotherapy. In addition, currently very popular targeted therapy and immunotherapy have been troubled by tumor drug resistance. In recent years, many studies have reported that ferroptosis is expected to be an important way to solve tumor drug resistance. However, the conventional ferroptosis process is affected by the active regulation of iron homeostasis and redox homeostasis by cells, and cannot cause cancer cells to undergo ferroptosis with a level of cancer therapeutic value. Based on a large number of studies on cancer gene therapy and the biological effects of iron nanomaterials, the present invention applies the principle of gene therapy technology to ferroptosis, and proposes gene interference-enhanced ferroptosis therapy (GIFT) and a new composition and anti-cancer treatment method. Experiments of the present invention demonstrate that all cancer cells tested are almost completely dead after 72 hours of treatment with the new method.

In summary, the present invention provides a composition for killing cancer cells by combining two biological and chemical materials, a gene interference carrier and iron nanoparticles, wherein the gene interference carrier is CRISPR/Cas13a controlled by a cancer cell-specific promoter DMP. Or microRNA expression vector, the Cas13a-gRNA or microRNA expressed by this vector can target and inhibit intracellular iron metabolism and the expression of reactive oxygen species-related genes, and the iron nanoparticles can be degraded to generate iron ions and increase the level of reactive oxygen species after entering cells. Under the combined action of the gene interference carrier and the iron nanoparticle, the invention can lead to a sharp increase in the levels of iron ions and reactive oxygen species in cancer cells and induce significant iron apoptosis in cancer cells. The proposed combination of gene interference vector and iron nanoparticles of the present invention can be used to prepare novel cancer therapeutic agents.

### DESCRIPTION OF THE ACCOMPANYING DRAWINGS

FIG. 1 is a schematic diagram of the functional DNA elements and sequences of the CRISPR/Cas13a expression vector, wherein in order to visualize the functional DNA elements and sequences of the vector, the plasmid in the figure is named pDMP-Cas13a-U6-gRNA, abbreviated as pDCUg; the figure shows that DMP controls Cas13a expression, while the U6 promoter controls gRNA expression; the vector is a backbone vector for constructing a CRISPR/Cas13a expression vector targeting specific genes.

FIG. 2 is a schematic diagram of the functional DNA elements and sequences of the microRNA expression vector, wherein the figure shows that DMP controls the expression of microRNAs, wherein the vector is a backbone vector and is used to construct a microRNA expression vector targeting specific genes.

FIG. 3 is a schematic diagram of the principle of gene interference ferroptosis therapy (GIFT), gene expression vector activated by NF-κB and Fe₃O₄ nanoparticles (FeNPs); NF-κB-activated gene expression vector consists of a NF-κB-specific promoter (DMP) and its downstream expressions, wherein the NF-κB-specific promoter consists of NF-κB decoy (Decoy) sequence and minimal promoter (Minimal Promoter, MP) sequence composition; wherein (A) Schematic diagram of the principle of GIFT based on CRISPR/Cas13a; U6-p is U6 promoter; gRNA is gRNA coding sequence; Cas13a, Cas13a coding sequence; (B) Schematic diagram of the principle of miRNA-based GIFT; (C) Quantitative PCR to detect the expression of NF-κB in different cell lines. ***, p<0.001.

FIG. 4 is a schematic diagram of the effect of FeNPs on cell viability. (A) Effects of FeNPs on the viability of three leukemia cells; three leukemia cells were treated with different concentrations of FeNPs; cell viability was detected by CCK-8 assay at various times after treatment; (B) The effect of FeNP on the viability of hepatoma cells and two normal cells (HL7702 and MRC-5); cells were treated with different concentrations of FeNPs; cell viability was detected by CCK-8 assay at various times after treatment.

FIG. 5 is a schematic diagram of the GIFT inhibition experiment of KG-1a cells. Cells were transfected with the various plasmids shown in the figure, cultured for 24 hours, and then cultured for an additional 72 hours in a medium with or without 50 μg/mL FeNPs. Cells were stained with acridine orange/ethidium bromide and imaged at various time points. Three leukemia cells were treated with various combinations of pDCUg or pDM vector and FeNPs. pDCUg refers to the plasmid of DMP-Cas13a-U6-gRNA, and pDM refers to the plasmid of DMP-miRNA. The plasmid vectors used include pDCUg-NT (gRNA does not target any transcript), pDCUg-hF (gRNA targets human FPN), pDCUg-hL (gRNA targets human Lcn2), pDCUg-hFL (gRNA targets human FPN) and Lcn2), pDCUg-mF (gRNA targets mouse FPN), pDCUg-mL (gRNA targets mouse Lcn2), pDCUg-mFL (gRNA targets mouse FPN and Lcn2), pDMNeg (miRNA does not target any transcription present), pDMhF (miRNA targeting human FPN), pDMhL (miRNA targeting human Lcn2), pDMhFL (miRNA targeting human FPN and Lcn2), pDMmF (miRNA targeting murine FPN), pDMmL (miRNA targeting murine Lcn2) and pDMmFL (miRNA targeting murine FPN and Lcn2). Cells were transfected with various plasmids and cultured for 24 h, followed by 72 h in medium with or without 50 μg/mL FeNPs. At each time point of FeNPs treatment, cells were stained with acridine orange/ethidium bromide and imaged. The figure shows only representative cell images of plasmids pDMNeg, pDMhFL, pDMmFL, pDCUg-NT, pDCUg-hFL and pDCUg-mFL treated with FeNPs for 72 hours.

FIG. 6 is a schematic diagram of the GIFT inhibition experiment of HL60 cells. Cells were transfected with the various plasmids shown in the figure, cultured for 24 hours, and then cultured for an additional 72 hours in medium with or without 50 μg/mL FeNPs. Cells were stained with acridine orange/ethidium bromide and imaged at various time points. The vector annotations are the same as in FIG. 5.

FIG. 7 is a schematic diagram of the GIFT inhibition experiment of WEHI-3 cells. Cells were transfected with the various plasmids shown in the figure, cultured for 24 hours, and then cultured for an additional 72 hours in medium with or without 50 μg/mL FeNPs. Cells were stained with acridine orange/ethidium bromide and imaged at various time points. The vector annotations are the same as in FIG. 5.

FIG. 8 is a schematic diagram of quantitative detection of apoptosis of three leukemia cells with GIFT inhibitory effect. Cells were treated with various combinations of plasmid vectors and FeNPs. Cells were collected 72 hours after FeNPs administration, and detected by Annexin V-FITC apoptosis detection kit and flow cytometry. The graph only shows the final statistical results. The treatments represented by the individual bars in each histogram on the left, from left to right, correspond to the various treatments from top to bottom in the annotation graph on the right. Representative flow cytometry images are shown in FIG. 9. All values are mean±s.e.m. where n=3. *, p<0.05; **, p<0.01; ***, p<0.001.

FIG. 9 is a schematic diagram showing the apoptosis of three leukemia cells treated with GIFT by flow cytometry and this figure shows a representative flow cytometry image.

FIG. 10 is a schematic diagram of the GIFT inhibition experiment of HepG2 cells. Cells were transfected with the various plasmids shown in the figure, cultured for 24 hours, and then cultured for an additional 72 hours in medium with or without 50 μg/mL FeNPs. Cells were stained with acridine orange/ethidium bromide and imaged at various time points. Cells were treated with various combinations of pDCUg or pDM vector and FeNPs. Plasmid vectors used included pDCUg-NT, pDCUg-hF, pDCUg-hL, pDCUg-hFL, pDMNeg, pDMhF, pDMhL and pDMhFL. Cells were transfected with various plasmids and cultured for 24 hours. Cells were then cultured with medium with or without 50 μg/mL FeNPs for an additional 72 hours. At various time points after FeNPs treatment, cells were stained with acridine orange/ethidium bromide and imaged.

FIG. 11 is a schematic diagram of the GIFT inhibition experiment of HL7702 cells; cells were transfected with various plasmids in the figure and cultured for 24 hours; the vector transfection of cells was the same as in FIG. 10; cells were induced with or without TNF-α (10 ng/mL) for 1 h; cells were then incubated with medium with or without 50 μg/mL FeNPs for an additional 72 h; and cells were stained with acridine orange/ethidium bromide and imaged at various time points.

FIG. 12 is a schematic diagram of the GIFT inhibition experiment of MRC-5 cells; cells were transfected with various plasmids in the figure and cultured for 24 hours; the vector transfection of cells was the same as in FIG. 10; cells were induced with or without TNF-α (10 ng/mL) for 1 h; cells were then incubated with medium with or without 50 μg/mL FeNPs for an additional 72 h; and cells were stained with acridine orange/ethidium bromide and imaged at various time points.

FIG. 13 is a schematic diagram of the apoptosis of HepG2, HL7702 and MRC-5 cells treated with GIFT by flow cytometry; cells were treated with various combinations of plasmid vectors and FeNPs; cells were collected 72 h after FeNPs administration and detected by flow cytometry with Annexin V-FITC Apoptosis Detection Kit; the graph only shows the final statistical results; the treatments represented by the individual bars in each histogram on the left, from left to right, correspond to the various treatments from top to bottom in the annotation graph on the right; representative flow cytometry images are shown in FIG. 14; all values are mean±s.e.m. wherein n=3. *, p<0.05; **, p<0.01; ***, p<0.001.

FIG. 14 is a schematic diagram of the apoptosis of HepG2, HL7702 and MRC-5 cells treated with GIFT by flow cytometry; this figure shows a representative flow cytometry image.

FIG. 15 is a schematic diagram of the GIFT inhibition experiment of HEK-293T cells; cells were transfected with the various plasmids shown in the figure and cultured for 24 hours; cells were then cultured for an additional 72 hours in medium with or without 50 μg/mL FeNPs; and cells were stained with acridine orange/ethidium bromide and imaged at various time points.

FIG. 16 is a schematic diagram of the GIFT inhibition experiment of A549 cells; cells were transfected with the various plasmids shown in the figure and cultured for 24 hours; cells were then cultured for an additional 72 hours in medium with or without 50 μg/mL FeNPs; and cells were stained with acridine orange/ethidium bromide and imaged at various time points.

FIG. 17 is a schematic diagram of the GIFT inhibition experiment of HT-29 cells; cells were transfected with the various plasmids shown in the figure and cultured for 24 hours; cells were then cultured for an additional 72 hours in medium with or without 50 μg/mL FeNPs; and cells were stained with acridine orange/ethidium bromide and imaged at various time points.

FIG. 18 is a schematic diagram of the GIFT inhibition experiment of PANC1 cells; cells were transfected with the various plasmids shown in the figure and cultured for 24 hours; cells were then cultured for an additional 72 hours in medium with or without 50 μg/mL FeNPs; and cells were stained with acridine orange/ethidium bromide and imaged at various time points.

FIG. 19 is a schematic diagram of the GIFT inhibition experiment of SKOV3 cells; cells were transfected with the various plasmids shown in the figure and cultured for 24 hours; cells were then cultured for an additional 72 hours in medium with or without 50 μg/mL FeNPs; and cells were stained with acridine orange/ethidium bromide and imaged at various time points.

FIG. 20 is a schematic diagram of the GIFT inhibition experiment of MDA-MB-453 cells; cells were transfected with the various plasmids shown in the figure and cultured for 24 hours; cells were then cultured for an additional 72 hours in medium with or without 50 μg/mL FeNPs; and cells were stained with acridine orange/ethidium bromide and imaged at various time points.

FIG. 21 is a schematic diagram of the GIFT inhibition experiment of C-33A cells; cells were transfected with the various plasmids shown in the figure and cultured for 24 hours; cells were then cultured for an additional 72 hours in medium with or without 50 μg/mL FeNPs, and cells were stained with acridine orange/ethidium bromide and imaged at various time points.

FIG. 22 is a schematic diagram of the GIFT inhibition experiment of BGC823 cells; cells were transfected with the various plasmids shown in the figure and cultured for 24 hours; cells were then cultured for an additional 72 hours in medium with or without 50 μg/mL FeNPs; and cells were stained with acridine orange/ethidium bromide and imaged at various time points.

FIG. 23 is a schematic diagram of the GIFT inhibition experiment of SGC7901 cells. Cells were transfected with the various plasmids shown in the figure and cultured for 24 hours; cells were then cultured for an additional 72 hours in medium with or without 50 μg/mL FeNPs; and cells were stained with acridine orange/ethidium bromide and imaged at various time points.

FIG. 24 is a schematic diagram of the GIFT inhibition experiment of MGC-803 cells. Cells were transfected with the various plasmids shown in the figure and cultured for 24 hours; cells were then cultured for an additional 72 hours in medium with or without 50 μg/mL FeNPs; and cells were stained with acridine orange/ethidium bromide and imaged at various time points.

FIG. 25 is a schematic diagram of the GIFT inhibition experiment of KYSE450 cells; cells were transfected with the various plasmids shown in the figure and cultured for 24 hours; cells were then cultured for an additional 72 hours in medium with or without 50 μg/mL FeNPs; and cells were stained with acridine orange/ethidium bromide and imaged at various time points.

FIG. 26 is a schematic diagram of the GIFT inhibition experiment of KYSE510 cells; and cells were transfected with the various plasmids shown in the figure and cultured for 24 hours; cells were then cultured for an additional 72 hours in medium with or without 50 μg/mL FeNPs; cells were stained with acridine orange/ethidium bromide and imaged at various time points.

FIG. 27 is a schematic diagram of the GIFT inhibition experiment of B16F10 cells; cells were transfected with the various plasmids shown in the figure and cultured for 24 hours; cells were then cultured for an additional 72 hours in medium with or without 50 μg/mL FeNPs; and cells were stained with acridine orange/ethidium bromide and imaged at various time points.

FIG. 28 is a schematic diagram of the GIFT inhibition experiment of Hepa1-6 cells; cells were transfected with the various plasmids shown in the figure and cultured for 24 hours; cells were then cultured for an additional 72 hours in medium with or without 50 μg/mL FeNPs; and cells were stained with acridine orange/ethidium bromide and imaged at various time points.

FIG. 29 is a schematic diagram of the knockdown effect of DMP-Cas13a/U6-gRNA and DMP-miR systems; cells were transfected with various vectors and incubated for 24 hours, then incubated with or without 50 μg/mL FeNPs; cells were detected 48 hours after FeNPs administration; (A) qPCR analysis of mRNA expression. (B) Western blot analysis of protein expression; representative images and quantitative optical densities are shown; all values are mean±s.e.m. where n=3. *, p<0.05; **, p<0.01; ***, p<0.001.

FIG. 30 is a schematic diagram showing the correlation between the increase of ROS generation and iron content and GIFT-induced apoptosis; cells were transfected with various plasmids and cultured for 24 hours, followed by an additional 48 hours with or without 50 μg/mL FeNPs; HL7702 and MRC-5 cells were cultured with or without induction of TNF-α (10 ng/mL) for 1 h before treatment with FeNPs; ROS changes and iron content were detected 48 hours after FeNPs administration; (A) Flow cytometric analysis of ROS levels; fluorescence shift and quantified fluorescence intensity are shown in the figure; treated cells were stained with DCFH-DA using a reactive oxygen species assay kit; ROS changes indicated by fluorescence shift were analyzed by flow cytometry; (B) Quantitative detection of iron content in cells under various treatments; all values are mean±s.e.m. where n=3. *, p<0.05; **, p<0.01; *** p<0.001.

FIG. 31 is a schematic diagram of the cytometry analysis of ROS levels in GIFT-treated cells; cells were transfected with the various plasmids shown in the figure and cultured for 24 hours; HL7702 and MRC-5 cells were induced with or without TNF-α (10 ng/mL) for 1 hour; then with or without 50 μg/mL FeNPs; the cells were cultured in the medium for an additional 48 hours; cells were harvested and stained with DCFH-DA using a reactive oxygen species assay kit, and flow cytometry was used to analyze ROS changes indicated by fluorescence shift.

FIG. 32 is a schematic diagram of in vitro assessment of rAAV. KG-1a, WEHI-3 and HL7702 cells were seeded into 24-well plates (1×105 cells/well) and cultured for 12 hours; cells were then transfected with various viruses in the figure at a dose of 1×105 vg per cell; transfected cells were cultured for 24 hours and then in medium containing or containing 50 μg/mL FeNPs for an additional 72 hours; cells were stained with acridine orange/ethidium bromide and imaged, and parallel cells were analyzed for cell viability with CCK-8; (A) Representative cell images. (B) Cell viability; and all values are mean±s.e.m. where n=3. *, p<0.05; **, p<0.01; ***, p<0.001.

FIG. 33 is a schematic diagram of the transfection of KG-1a cells with Fe nanocarriers (FeNCs) loaded with various plasmids; cells (1×105) were seeded into 24-well plates overnight before transfection; cells were treated with FeNCs (0.5 μg) loaded with 500 ng of each plasmid according to the manufacturer's instructions; the transfected cells were cultured for 24 hours, and then the cells were incubated with medium with or without 50 μg/mL FeNPs for an additional 72 hours; all cells were stained with acridine orange/ethidium bromide at 24, 48 and 72 hours after FeNPs administration and imaged under a fluorescence microscope.

FIG. 34 is a schematic diagram of HepG2 cells transfected with Fe nanocarriers (FeNCs) loaded with various plasmids; cells (1×105) were seeded into 24-well plates overnight before transfection; cells were treated with FeNCs (0.5 μg) loaded with 500 ng of each plasmid according to the manufacturer's instructions; the transfected cells were cultured for 24 hours, and then the cells were incubated with medium with or without 50 μg/mL FeNPs for an additional 72 hours; all cells were stained with acridine orange/ethidium bromide at 24, 48 and 72 hours after FeNPs administration and imaged under a fluorescence microscope.

FIG. 35 is a schematic diagram of the transfection of KG-1a cells by two kinds of Fe nanocarriers (FeNCs) loaded with various plasmids; cells (1×105) were seeded into 24-well plates overnight before transfection; cells were treated with 50 g/mL FeNCs (FeNCs-1 and FeNCs-2) loaded with various plasmids; all cells were cultured for an additional 72 hours; all cells were stained with acridine orange/ethidium bromide at 24, 48 and 72 hours after FeNCs administration and imaged under a fluorescence microscope; the pDMFL was mixed with FeNCs-1/FeNCs-2 according to the instructions to form FeNCs-1/FeNCs-2 carrying plasmid pDMFL (denoted as FeNCs-1@pDMFL/FeNCs-2@pDMFL); FeNCs-1@pDMFL/FeNCs-2@pDMFL were added to cells immediately or added to cells after 24 hours (represented as FeNCs-1@pDMFL24h/FeNCs-2@pDMFL24h); FeNCs-1/FeNCs-2 represent two kinds of FeNCs.

FIG. 36 is a schematic diagram of the in vivo antitumor effect of viral vector-based GIFT; (A) Tumor photos of the first and second batch of animal experiments; (B) Changes in tumor volume before and after treatment; (C) Abundance of viral DNA in various tissues; (D) Ct values of Cas13a mRNA detected by qPCR in various tissues; (E) Relative expression (RQ) of FPN mRNA in various tissues; (F) Relative expression (RQ) of Lcn2 mRNA in various tissues; All values are mean±s.e.m. where n=3. *, p<0.05; **, p<0.01; ***, p<0.001.

FIG. 37 is a schematic diagram of the in vivo antitumor effect of plasmid-loaded iron nanoparticles-based GIFT; (A) Tumor photos of the third batch of animal experiments; (B) Changes in tumor volume before and after treatment; (C) Abundance of plasmid DNA in various tissues; (D) Ct values of Cas13a mRNA detected by qPCR in various tissues; (E) Relative expression (RQ) of FPN mRNA in various tissues; (F) Relative expression (RQ) of Lcn2 mRNA in various tissues; and all values are mean±s.e.m. where n=3. *, p<0.05; **, p<0.01; ***, p<0.001.

FIG. 38 is a schematic diagram of the GIFT inhibition experiment of KG-1a cells; cells were transfected with the various plasmids shown in the figure and cultured for 24 hours; cells were then cultured for an additional 72 hours in medium with or without 50 μg/mL FeNPs; cells were stained with acridine orange/ethidium bromide and imaged at various time points; plasmids to treat cells include pDMhFSP1-1 (miFSP1-1), pDMhFSP1-2 (miFSP1-2), pDMhFTH1-1 (mihFTH1-1), pDMhFTH1-2 (miFTH1-2), pDMhGPX4-1 (mi GPX4-1), pDMhGPX4-2 (miGPX4-2), pDMhNRF2-1 (miNRF2-1), pDMhNRF2-2 (miNRF2-2), pDMhSLC7A11-1 (miSLC7A11-1) and pDMhSLC7A11-2 (miSLC7A11-2) (each abbreviation of the vector is added in the parentheses).

FIG. 39 is a schematic diagram of the GIFT inhibition experiment of HepG2 cells; cells were transfected with the various plasmids shown in the figure and cultured for 24 hours; cells were then cultured for an additional 72 hours in medium with or without 50 μg/mL FeNPs; cells were stained with acridine orange/ethidium bromide and imaged at various time points; and the plasmids used to treat the cells are the same as in FIG. 38.

FIG. 40 is a schematic diagram of the GIFT inhibition experiment of HL7702 cells; cells were transfected with the various plasmids shown in the figure and cultured for 24 hours; cells were then cultured for an additional 72 hours in medium with or without 50 μg/mL FeNPs; cells were stained with acridine orange/ethidium bromide and imaged at various time points; and the plasmids used to treat the cells are the same as in FIG. 38.

FIG. 41 is a schematic diagram of the GIFT inhibition experiment of BGC823 cells; cells were transfected with the various plasmids shown in the figure and cultured for 24 hours; cells were then cultured for an additional 72 hours in medium with or without 50 μg/mL FeNPs; cells were stained with acridine orange/ethidium bromide and imaged at various time points; and the plasmids used to treat the cells are the same as in FIG. 38.

FIG. 42 is a schematic diagram of the GIFT inhibition experiment of GES-1 cells; cells were transfected with the various plasmids shown in the figure and cultured for 24 hours; cells were then cultured for an additional 72 hours in medium with or without 50 μg/mL FeNPs; cells were stained with acridine orange/ethidium bromide and imaged at various time points; the plasmids used to treat the cells are the same as in FIG. 38.

FIG. 43 is a schematic diagram of the in vitro antitumor effect of GIFT targeting other genes; using pDMP-miR vectors targeting 5 genes (FSP1, FTH1, GPX4, NRF2, and SLC7A11), including pDMhFSP1-1 (miFSP1-1), pDMhFSP1-2 (miFSP1-2), pDMhFTH1-1 (mihFTH1-1), pDMhFTH1-2 (miFTH1-2), pDMhGPX4-1 (mi GPX4-1), pDMhGPX4-2 (miGPX4-2), pDMhNRF2-1 (miNRF2-1), pDMhNRF2-2 (miNRF2-2), pDMhSLC7A11-1 (miSLC7A11-1) and pDMhSLC7A11-2 (miSLC7A11-2) (abbreviations for each vector in parentheses), transfected 5 cells, 24 hours later; re-cultured with medium with or without 50 μg/mL FeNPs cells for 72 hours. Cell viability was analyzed with CCK-8 at different time points; and all values are mean±s.e.m. where n=3. *, p<0.05; **, p<0.01; ***, p<0.001; ****, p<0.0001. The three data columns of each treatment in the figure are the detection data of 24 hours, 48 hours, and 72 hours from left to right.

### DESCRIPTION OF DETAIL EMBODIMENTS

The present invention will be further described below with reference to the accompanying drawings and embodiments.

### EXAMPLES

Gene-interfering ferroptosis therapy (GIFT) inhibits cancer cell growth in vitro and in vivo.

1. Materials and Methods

1.1 Vector Construction

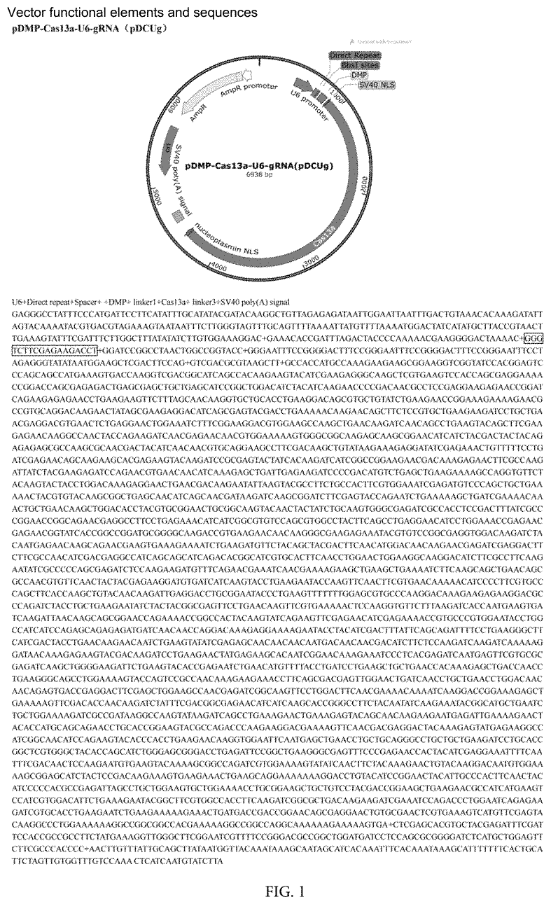

The decoy minimal promoter (DMP), a chemically synthesized NF-κB-specific promoter containing an NF-κB response sequence and a minimal promoter sequence, was cloned into pMD19-T simple (TAKARA) to give pMD19-T-DMP; the human codon-optimized Cas13a coding sequence was amplified from pC013-Twinstrep-SUMO-huLwCas13a (Addgene) by PCR, and the amplified product was cloned into pMD19-T-DMP to obtain pMD19-T-DMP-Cas13a; the U6 promoter sequence and the direct repeat sequence (guide RNA, gRNA) of Cas13a separated by the BbsI restriction site were chemically synthesized and cloned into pMD19-T-DMP-Cas13a, respectively; obtain pDMP-Cas13a-U6-gRNA (referred to as pDCUg for short), the DNA sequence of its functional element is shown in SEQ ID NO.1 and FIG. 1, this vector is a skeleton vector for constructing an expression vector pdcug-x targeting different gene (x) transcripts.

gRNAs targeting no transcript (NT), human or murine ferroportin (FPN), and Lipocalin 2 (Lcn2) transcripts were designed by CHOPCHOP (http://chopchop.cbu.uib.no/). Complementary oligonucleotides containing a 28 bp gRNA target-specific region and two flanking BbsI sites were chemically synthesized, annealed into double-stranded oligonucleotides, and then cloned into pDCUg by BbsI digestion and ligase. The ligation reaction (10 μL) consisted of 10 units of BbsI enzyme (NEB), 600 units of T4 DNA ligase (NEB), 1×T4 DNA ligase buffer, 1 nM double-stranded oligonucleotide, and 50 ng of pDCUg. The ligation reaction was run on a PCR cycler with the following temperature-controlled program: 10 cycles of 37° C. for 5 minutes and 16° C. for 10 minutes, 37° C. for 30 minutes and 80° C. for 5 minutes. The resulting plasmids were named pDCUg-NT, pDCUg-hFPN/pDCUg-mFPN, pDCUg-hLcn2/pDCUg-mLcn2, respectively. Due to the difference between human and mouse gene sequences, vectors targeting human FPN (hFPN) and Lcn2 (hLcn2) genes, and mouse FPN (mFPN) and Lcn2 (mLcn2) genes were constructed respectively (h, human; m, mouse). In addition, a pDCUg vector targeting both FPN and Lcn2 genes was constructed, named pDCUg-hFL/pDCUg-mFL. Cas13a gRNAs targeting all genes of interest are listed in Table 1.

TABLE 1
gRNA target sequences of Cas13a

| Name | Sequence (5′→3′) | PFS |
|---|---|---|
| Human FPN | CACCGCAAAGTGCCACATCCGATCTCCC | T |
| guide | | |
| Human Lcn2 | TAACTCTTAATGTTGCCCAGCGTGAACT | C |
| guide | | |
| Mouse FPN | TTATTCCAGTTATTGCTGATGCTCCCAT | T |
| guide | | |
| Mouse Len2 | TTGGTCGGTGGGGACAGAGAAGATGATG | T |
| guide | | |
| No transcript | TAGATTGCTGTTCTACCAAGTAATCCAT | N/A |
| guide | | |

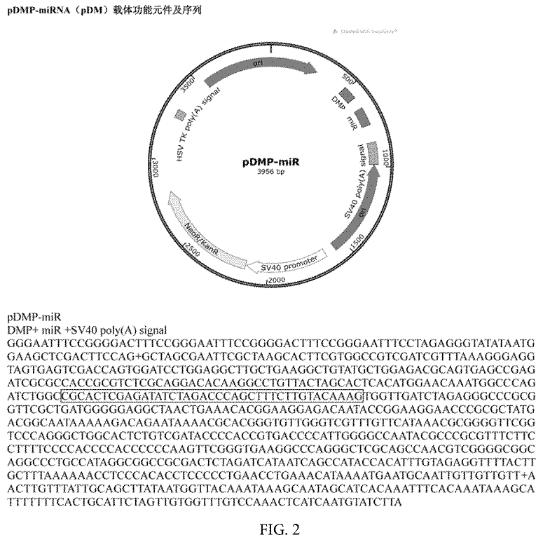

The CMV promoter in the pCMV-miR vector was replaced with the DMP promoter to construct a universal miRNA expression vector pDMP-miR, the DNA sequence of its functional elements is shown in SEQ ID NO. 2 and FIG. 2. Among them, pCMV-miR was previously constructed by the inventor's laboratory (Int. J. Biochem. Cell. Biol. 2018, 95:43-52), which contains the CMV promoter and its downstream miR backbone sequence. miRNAs targeting human or murine FPN and Lcn2 were designed using the BLOCK-iT™ RNAi Designer (https://maidesigner.thermofisher.com/rnaiexpress/) program, and the corresponding oligonucleotides were synthesized by Sangon Biotech. The synthesized oligonucleotides were denatured and reannealed to obtain double-stranded oligonucleotides, which were then ligated with the linear pDMP-miR vector cut with BsmBI to generate miRNA expression vectors targeting FPN and Lcn2 genes, named pDMP, respectively -miR-hFPN/pDMP-miR-mFPN (pDMhF/pDMmF for short) and pDMP-miR-hLcn2/pDMP-miR-mLcn2 (pDMhL/pDMmL for short) Use the symbol “/” to mean “or”). The detection vector was amplified by PCR and verified by DNA sequencing. In addition, a miRNA expression vector that can simultaneously express (i.e. co-express) both FPN and Lcn2 was constructed and named as pDMP-miR-hFPN-DMP-miR-hLcn2/pDMP-miR-mFPN-DMP-miR-mLcn2 (abbreviated as pDMhFL/pDMmFL). In a similar manner, miR-Neg double-stranded oligonucleotides were synthesized and prepared from the sequence of plasmid pcDNA™6.2-GW/EmGFP-miR-Neg, and ligated into the pDMP-miR vector to generate pDMP-miR-Neg (abbreviated as pDMNeg), this vector was used as a negative control vector. The target sequences and chemically synthesized oligonucleotide sequences used to construct pDMIP-miR vectors targeting individual genes are shown in Table 2.

The same method was used to design and construct pDMP-miR vectors targeting other five genes, namely FSP1, FTH1, GPX4, NRF2 and SLC7A11; and miRNAs targeting two targets were designed for each gene. The constructed vectors were named pDMhFSP1-1, pDMhFSP1-2, pDMhFTH1-1, pDMhFTH1-2, pDMhGPX4-1, pDMhGPX4-2, pDMhNRF2-1, pDMhNRF2-2, pDMhSLC7A11-1 and pDMhSLC7A11-2, respectively. The target sequences and chemically synthesized oligonucleotide sequences used to construct pDMP-miR vectors targeting individual genes are shown in Table 2.

TABLE 2
Oligonucleotides used to construct pDMP-miRNA vectors targeting
different genes

| Name | Sequence (5′→3′) |
|---|---|
| Human miR-FPN-F | TGCTGTCTACCTGCAGCTTACATGATGTTTTGGCCACTGACTGACATCATGTACTGCAGGTAGA |
| Human miR-FPN-R | CCTGTCTACCTGCAGTACATGATGTCAGTCAGTGGCCAAAACATCATGTAAGCTGCAGGTAGAC |
| Murine miR-FPN-F | TGCTGTATACAGACTCACTGATTTGCGTTTTGGCCACTGACTGACGCAAATCAGAGTCTGTATA |
| Murine miR-FPN-R | CCTGTATACAGACTCTGATTTGCGTCAGTCAGTGGCCAAAACGCAAATCAGTGAGTCTGTATAC |
| Human miR-Lcn2-F | TGCTGTAATGTTGCCCAGCGTGAACTGTTTTGGCCACTGACTGACAGTTCACGGGGCAACATTA |
| Human miR-Lcn2-R | CCTGTAATGTTGCCCCGTGAACTGTCAGTCAGTGGCCAAAACAGTTCACGCTGGGCAACATTAC |
| Murine miR-Lcn2-F | TGCTGTCAAGTTCTGAGTTGAGTCCTGTTTTGGCCACTGACTGACAGGACTCATCAGAACTTGA |
| Murine miR-Lcn2-R | CCTGTCAAGTTCTGATGAGTCCTGTCAGTCAGTGGCCAAAACAGGACTCAACTCAGAACTTGAC |
| miR-Neg-F | TGCTGAAATGTACTGCGCGTGGAGACGTTTTGGCCACTGACTGACGTCTCCACGCAGTACATTT |
| miR-Neg-R | CCTGAAATGTACTGCGTGGAGACGTCAGTCAGTGGCCAAAACGTCTCCACGCGCAGTACATTTC |
| Human miR-Fsp1-1-F | TGCTGCAAACAAACAAATAAAGTGGAGTTTTGGCCACTGACTGACTCCACTTTTTGTTTGTTTG |
| Human miR-Fsp1-l-R | CCTGCAAACAAACAAAAAGTGGAGTCAGTCAGTGGCCAAAACTCCACTTTATTTGTTTGTTTGC |
| Human miR-Fsp1-2-F | TGCTGTAAACAAACAAACAAATAAAGGTTTTGGCCACTGACTGACCTTTATTTTTGTTTGTTTA |
| Human miR-Fsp1-2-R | CCTGTAAACAAACAAAAATAAAGGTCAGTCAGTGGCCAAAACCTTTATTTGTTTGTTTGTTTAC |
| Human miR-Fth1-1-F | TGCTGATCCCAAGACCTCAAAGACAAGTTTTGGCCACTGACTGACTTGTCTTTGGTCTTGGGAT |
| Human miR-Fth1-1-R | CCTGATCCCAAGACCAAAGACAAGTCAGTCAGTGGCCAAAACTTGTCTTTGAGGTCTTGGGATC |
| Human miR-Fth1-2-F | TGCTGTAAGGAATCTGGAAGATAGCCGTTTTGGCCACTGACTGACGGCTATCTCAGATTCCTTA |
| Human miR-Fth1-2-R | CCTGTAAGGAATCTGAGATAGCCGTCAGTCAGTGGCCAAAACGGCTATCTTCCAGATTCCTTAC |
| Human miR-Gpx4-1-F | TGCTGTTCAGTAGGCGGCAAAGGCGGGTTTTGGCCACTGACTGACCCGCCTTTCGCCTACTGAA |
| Human miR-Gpx4-1-R | CCTGTTCAGTAGGCGAAAGGCGGGTCAGTCAGTGGCCAAAACCCGCCTTTGCCGCCTACTGAAC |
| Human miR-Gpx4-2-F | TGCTGAGGAACTGTGGAGAGACGGTGGTTTTGGCCACTGACTGACCACCGTCTCCACAGTTCCT |
| Human miR-Gpx4-2-R | CCTGAGGAACTGTGGAGACGGTGGTCAGTCAGTGGCCAAAACCACCGTCTCTCCACAGTTCCTC |
| Human miR Nrf2-1-F | TGCTGTACTGATTCAACATACTGACAGTTTTGGCCACTGACTGACTGTCAGTATTGAATCAGTA |
| Human miR-Nrf2-1-R | CCTGTACTGATTCAATACTGACAGTCAGTCAGTGGCCAAAACTGTCAGTATGTTGAATCAGTAC |
| Human miR-Nrf2-2-F | TGCTGTTTACACTTACACAGAAACTAGTTTTGGCCACTGACTGACTAGTTTCTGTAAGTGTAAA |
| Human miR-Nrf2-2-R | CCTGTTTACACTTACAGAAACTAGTCAGTCAGTGGCCAAAACTAGTTTCTGTGTAAGTGTAAAC |
| Human miR-SLC7A11-1-F | TGCTGAAATGATACAGCCTTAACACAGTTTTGGCCACTGACTGACTGTGTTAACTGTATCATTT |
| Human miR-SLC7A11-1-R | CCTGAAATGATACAGTTAACACAGTCAGTCAGTGGCCAAAACTGTGTTAAGGCTGTATCATTTC |
| Human miR-SLC7A11-2-F | TGCTGTTGAGTTGAGGACCAGTTAGTGTTTTGGCCACTGACTGACACTAACTGCCTCAACTCAA |
| Human miR-SLC7A11-2-R | CCTGTTGAGTTGAGGCAGTTAGTGTCAGTCAGTGGCCAAAACACTAACTGGTCCTCAACTCAAC |

DCUg-NT/hFL/mFL and DMNeg/DMhFL/DMmFL sequences were amplified by PCR from pAAV-DCUg-NT/hFL/mFL and pAAV-DMNeg/DMhFL/DMmFL, respectively. Using MluI (upstream) and XbaI (downstream) restriction sites, the DCUg-NT/hFL/mFL and DMNeg/DMhFL/DMmFL sequences were cloned into pAAV-MCS (VPK-410, Stratagene) to construct pAAV-DCUg-NT/hFL/mFL and pAAV-DMNeg/DMhFL/DMmFL vectors, respectively.

1.2. Nanoparticles, Cells and Culture

DMSA-coated Fe₃O₄ magnetic nanoparticles (FeNPs) and polyethylenimine (PEI)-modified ferric oxide nanoparticles (FeNCs) were purchased from Nanjing Dongna Biotechnology Co., Ltd.

Cells used in the present invention include KG-1a (human acute myeloid leukemia cells), HL60 (human amyloid acute leukemia cells), WEHI-3 (mouse acute monocytic leukemia cells), HepG2 (human liver cancer cells) cells), A549 (human lung cancer cells), HT-29 (human colon cancer cells), C-33A (human cervical cancer cells), SKOV3 (human ovarian cancer cells), PANC-1 (human pancreatic cancer cells), MDA-MB-453 (human breast cancer cells), BGC-823/MGC-803/SGC-7901 (human gastric adenocarcinoma cells), KYSE450/KYSE510 (human esophageal cancer cells), Hepa1-6 (mouse liver cancer cells), B16F10 (mouse melanoma cells), HEK-293T (human fetal kidney cells), HL7702 (human normal hepatocytes), MRC5 (human embryonic fibroblasts) and GES-1 (human normal gastric mucosal epithelial cells). Three leukemia cell lines KG-1a, HL60 and WEHI-3 were cultured in IMEM medium (Gibco). HEK-293T, HepG2, Hepa1-6, C-33A, PANC-1, MDA-MB-453, B16F10, MRC-5, GES-1 cells were cultured with DMEM medium (Gibco). A549, HT-29, SKOV-3, BGC-823/MGC-803/SGC-7901, KYSE450/KYSE510 and HL7702 cells were cultured in RPMI 1640 medium (Gibco). All three media were supplemented with 10% fetal bovine serum (HyClone), 100 units/mL penicillin (Thermo Fisher Scientific), and 100 μg/mL streptomycin (Thermo Fisher Scientific). Cells were incubated at 37° C. in a humidified incubator with 5% CO₂.

1.3. Cytotoxicity of FeNPs

Determine the optimal dose of nanoparticles. In vitro cytotoxicity of FeNPs was performed using CCK-8 assay. KG-1a, HL60, WEHI-3, HepG2, HL7702 and MRC-5 cells were seeded into 96-well plates at a density of 5000 cells/well, respectively. Cells were cultured overnight and treated multiple times with various concentrations (0 μg/mL, 30 μg/mL, 50 μg/mL, 100 μg/mL, 150 μg/mL, 200 μg/mL, 250 μg/mL) of FeNPs. Six groups of cells were used for each treatment, with four replicates per group. 10 μL of Cell Counting Kit-8 (CCK-8) solution (BS350B, Biosharp) was added to each well at different time points after treatment (0 d, 1 d, 2 d, 3 d, 4 d and 5 d) middle. After an additional 1 h incubation at 37° C., the optical density at 450 nm was measured using a microplate reader (BioTek).

1.4. Treatment of Cells with Gene Regulatory Tools and FeNPs

Cells were transfected with plasmids using Lipofectamine 2000 (Thermo Fisher Scientific) following the manufacturer's instructions. Briefly, cells (1×105 cells/well) were seeded into 24-well plates overnight before transfection. Cells were then transfected with 500 ng of various plasmids, including pDCUg-NT, pDCUg-hFPN/pDCUg-mFPN, pDCUg-hLcn2/pDCUg-mLcn2, pDCUg-hFL/pDCUg-mFL, pDMNeg, pDMhF/pDMmF, pDMhL/pDMmFL. Transfected cells were incubated for 24 hours, then incubated with or without 50 μg/mL FeNPs, and cells were incubated for an additional 72 hours. For HL7702 and MRC5, cells were first incubated with or without TNF-α (10 ng/mL) for 1 h before treatment with FeNPs. 24h, 48h and 72h after FeNPs administration, all cells were stained with acridine orange/ethidium bromide according to the manufacturer's instructions. Cells were imaged under a fluorescence microscope (IX51, Olympus) to observe the number of live and dead cells. To quantify apoptosis, cells were collected 72 h after administration of FeNPs and detected using the Annexin V-FITC Apoptosis Detection Kit (BD, USA) according to the manufacturer's instructions. Fluorescence intensity of cells was quantified with a CytoFLEX LX flow cytometer (Beckman).

1.5. Analysis of Reactive Oxygen Species Production

Cells were treated with FeNPs as described in step 1.4. Briefly, cells were seeded in 24-well plates (1×105 cells/well) and cultured overnight. Cells were then transfected with 500 ng of various plasmids, including pDCUg-NT, pDCUg-hFL/pDCUg-mFL, pDMNeg, and pDMhFL/pDMmFL. Transfected cells were cultured for 24 h, followed by an additional 48 h with or without incubation with FeNPs at 50 μg/mL. Treated cells were stained with 2′,7′-dichlorodihydrofluorescein diacetate (DCFH-DA) using a reactive oxygen species assay kit (Beyotime) according to the manufacturer's instructions. Fluorescence shifts were indicative of ROS changes when analyzed on a CytoFLEX LX flow cytometer (Beckman).

1.6. Iron Content Determination

Cells were processed as in step 1.5. Intracellular iron was determined by complete digestion of cells 48 hours after FeNPs administration. Cells were washed with PBS (pH 7.0), collected and counted. Cells were then pelleted by centrifugation, resuspended in 50 μL of 5M hydrochloric acid, and incubated at 60° C. for 4 hours. Cells were centrifuged again and the supernatant was transferred to a 96-well plate. Add 50 μL of freshly prepared detection reagent (0.08% K2S2O8, 8% KSCN and 3.6% HCl in water) to each well and incubate the microplate for 10 min at room temperature. Absorbance at 490 nm was measured using a microplate reader (BioTek). Iron content was determined by the absorbance obtained after normalization with a standard curve generated from a FeCl₃ standard solution. Iron is reported as the mean iron content per cell, calculated as the mean divided by the number of cells in each sample. Three replicate wells were set up for each experiment and repeated at least six times.

1.7. Western Blot Analysis

Cells were seeded into 6-well plates (2×105 cells/well) and grown overnight. Cells in each well were transfected with 1000 ng of pDCUg-NT, pDCUg-hFL, pDMNeg, and pDMhFL plasmid DNA, respectively. Forty-eight hours after transfection, whole cell extracts were prepared using a phosphoprotein extraction kit (SA6034-100T, Signalway Antibody, USA) according to the manufacturer's instructions. The protein lysate (20 μg/sample) was resolved by SDS-PAGE, and the target protein was detected by Western blot (WB). The antibodies used to detect the target protein in WB were: GAPDH rabbit monoclonal antibody (ab181602, Abcam, UK), SLC40A1 rabbit polyclonal antibody (ab58695, Abcam, UK), Lipocalin-2 rabbit polyclonal antibody (ab63929, Abcam, UK). The secondary antibody was IRDye 800CW-labeled goat anti-rabbit IgG (Licor). PVDF blots were imaged using an Odyssey infrared fluorescence imaging system (Licor) and the fluorescence intensity was quantified.

1.8. Virus Preparation

HEK293T cells were seeded into 75 cm² flasks (5×106 cells/flask) and cultured overnight. Cells were then transfected using Lipofectamine 2000 following the manufacturer's instructions, and the transfected DNA was two helper plasmids, pHelper and pAAV-RC (Stratagene), and one pAAV plasmid, including pAAV-DCUg-NT, pAAV-DCUg-hFL/pAAV-DCUg-mFL, pAAV-DMNeg and pAAV-DMhFL/pAAV-DMmFL. Cells were cultured for an additional 72 hours after transfection. The virus was then collected and purified as described in the literature (Gene Therapy, 2020, DOI: 10.1038/s41434-020-0128-x). AAV titers were determined by qPCR using primers AAV-F/R (Table 3). The quantified virus was aliquoted and stored at −80° C. until use. The obtained viruses were named rAAV-DCUg-NT, rAAV-DCUg-hFL/rAAV-DCUg-mFL, rAAV-DMNeg and rAAV-DMhFL/rAAV-DMmFL.

1.9. Virus Assessment

KG-1a, WEHI-3 and HL7702 cells were seeded into 24-well plates (1×105 cells/well) and cultured for 12 hours. Cells were then transfected with rAAV-DCUg-NT, rAAV-DCUg-hFL/rAAV-DCUg-mFL, rAAV-DMNeg, and rAAV-DMhFL/rAAV-DMmFL, respectively, at a virus dose of 1×105 vg per cell. Transfected cells were cultured for 24 hours and then incubated with or without 50 μg/mL FeNPs for an additional 72 hours. All cells were stained and imaged with acridine orange/ethidium bromide. Cell viability was detected using the CCK-8 assay (BS350B, Biosharp).

1.10. Iron Nanocarriers (FeNCs)-Based GIFT Inhibits Cancer Cells

In order to verify whether PEI-modified Fe₃O₄ iron nanoparticles (FeNCs) can be used as a delivery vehicle for gene interference carriers to introduce gene interference carriers into cells, and play the role of GIFT in inhibiting cancer cells together with iron nanoparticles as gene delivery vehicles, two studies were conducted. cell experiments. In order to distinguish iron nanoparticles as gene delivery vehicles from common iron nanoparticles (FeNPs) used above, iron nanoparticles as gene delivery vehicles are defined as iron nanocarriers (FeNCs). Two batches of FeNCs, named FeNCs-1 and FeNCs-2, were used in the experiments.

In the first FeNCs-based GIFT inhibition of cancer cells, various plasmids (including pDCUg-NT, pDCUg-hFL, pDMNeg, and pDMhFL) were mixed with FeNCs-1 (1 μg DNA/μg FeNCs-1), to prepare FeNCs loaded with plasmid DNA, namely FeNCs-1@pDCUg-NT, FeNCs-1@pDCUg-hFL, FeNCs-1@pDMNeg and FeNCs-1@pDMhFL. Cells were seeded into 24-well plates (1×105 cells/well) and cultured overnight. Cells were then treated with FeNCs with or without 0.5 μg plasmid DNA loaded and plasmid DNA alone for 24 hours; cells were then cultured with or without 50 μg/mL FeNPs for 72 hours. Differentially treated cells were stained with acridine orange/ethidium bromide and imaged at different time points (24 hours, 48 hours and 72 hours).

In the second FeNCs-based GIFT inhibition of cancer cells, two plasmids (pDMNeg and pDMhFL) were mixed with FeNCs-1 and FeNCs-2 (1 μg DNA/μg FeNCs-1) according to the manufacturer's instructions to prepare the load FeNCs of plasmid DNA, namely FeNCs-1@pDMhFL and FeNCs-2@pDMhFL. The prepared FeNCs-1@pDMhFL and FeNCs-2@pDMhFL were used to treat cells immediately or left at room temperature for 24 hours before treatment. Cells were seeded into 24-well plates (1×105 cells/well) and cultured overnight. Cells were then incubated with or without 50 μg/mL FeNCs (FeNCs alone or FeNCs loaded with plasmid DNA) for an additional 72 hours. Differentially treated cells were stained with acridine orange/ethidium bromide and imaged at different time points (24 hours, 48 hours and 72 hours).

1.11. Animal Studies

Four-week-old BALB/c female mice with an average body weight of 20 g were purchased from Changzhou Cavens Laboratory Animal Co. Ltd., China. All animal experiments in the present invention followed the guidelines and ethics of the Animal Care and Use Committee of Southeast University (Nanjing, China). The tumor-bearing mouse model was established by subcutaneously transplanting 1×107 WEHI-3 cells into the inner thigh of BALB/c female mice; after feeding for 1 week, the tumor size was measured in vivo with a precision caliper. Tumor volume was calculated using the formula V=(ab2)/2, wherein a is the longest tumor diameter and b is the shortest tumor diameter. Considering that the number of animals and cells required would be too large to manage if all experimental groups were performed at the same time, animal experiments were performed in three batches.

In the first batch of animal experiments, tumor-bearing mice were randomly divided into six treatment groups (PBS, n=6; FeNPs, n=6; rAAV-DCUg-NT, n=6; rAAV-DCUg-NT+FeNPs, n=6; rAAV-DCUg-mFL, n=6; rAAV-DCUg-mFL+FeNPs, n=7) (n is the number of mice). Each group of mice was injected intravenously with PBS (pH7.0), rAAV-DCUg-NT, rAAV-DCUg-NT, rAAV-DCUg-mFL, rAAV-DCUg-mFL, respectively. All viruses were injected at a dose of 1×1010 vg/mouse. On the second day, three of the groups (FeNPs, rAAV-DCUg-NT+FeNPs and rAAV-DCUg-mFL+FeNPs) were intravenously injected with FeNPs at a dose of 3 mg/kg body weight. On day 7 after FeNPs injection, mice were euthanized and photographed, then tumors were excised, and tumor sizes were measured and calculated as described above. Mice were dissected and various tissues (including heart, liver, spleen, lung, kidney, and tumor tissues) were collected and cryopreserved in liquid nitrogen.

In the second batch of animal experiments, tumor-bearing mice were randomly divided into five treatment groups (FeNPs, n=6; rAAV-DMNeg, n=6; rAAV-DMmFL, n=7; rAAV-DMNeg+FeNPs, n=7; rAAV-DMmFL+FeNPs, n=6). Then each group of mice was injected intravenously with FeNPs, rAAV-DMNeg, rAAV-DMmFL, rAAV-DMNeg+FeNPs, rAAV-DMmFL+FeNPs, respectively. The injection doses of all viruses and FeNPs were the same as the first batch of animal experiments, but in this batch of animal experiments, rAAV (1×1010 vg/mouse) was first mixed with FeNPs (3 mg/kg body weight), and then injected intravenously at one time mice. On day 7 post-injection, mice were euthanized and photographed, then tumors were dissected, and tumor sizes were measured and calculated as described above. The mice were dissected, and various tissues were collected and cryopreserved in liquid nitrogen.

In the third batch of animal experiments, tumor-bearing mice were randomly divided into six treatment groups (PBS, n=6; FeNCs, n=6; pAAV-DMNeg+FeNCs, n=6; pAAV-DMmFL+FeNCs, n=7; pAAV-DCUg-NT+FeNCs, n=6; pAAV-DCUg-mFL+FeNCs, n=7). Then each group of mice was injected intravenously with PBS (pH7.0), FeNCs, pAAV-DMNe+FeNCs, pAAV-DMmFL+FeNCs, pAAV-DCUg-NT+FeNCs, pAAV-DCUg-mFL+FeNCs, respectively. The doses of different plasmids and FeNCs were 2 mg/kg body weight and 3 mg/kg body weight, respectively. On day 7 after FeNCs injection, mice were euthanized and photographed, then tumors were dissected and tumor sizes were measured and calculated as described above. Mice were dissected and various tissues were collected and cryopreserved in liquid nitrogen.

1.12. Quantitative PCR

Total RNA was isolated from cells or mouse tissues after 48 h incubation with FeNPs using TRIzol™ (Invitrogen) according to the manufacturer's instructions. cDNA was prepared using the FastKing RT kit (TIANGEN) according to the manufacturer's instructions. Genomic DNA (gDNA) was extracted from various tissues of mice using the TIANamp Genomic DNA Kit (TIANGEN). Target genes were amplified from cDNA and gDNA by qPCR using Hieff qPCR SYBR Green Master Mix (Yeasen). Triplicate samples for each treatment were evaluated on an ABI Step One Plus (Applied Biosystems). Relative mRNA transcript levels were compared to the GADPH internal reference and calculated as relative quantity (RQ) according to the following equation: RQ=2−ΔΔCt. Viral DNA abundance was normalized to GADPH internal reference and calculated according to the following formula: RQ=2−ΔCt. Cas13a mRNA expression levels are shown as Ct values. All experiments were performed in triplicate and repeated at least three times.

The expression of NF-κB RelA/p65 in cells was detected by quantitative PCR (qPCR) using primers Human/Murine RelA-F/R and Human/Murine GAPDH-F/R. Results were normalized to GAPDH and analyzed by the 2−ΔCt method. According to melting curve analysis, all qPCR primers have amplification specificity, and their sequences are shown in Table 3.

Table 3
qPCR guide

| Name | Sequence (5′→3′) |
|---|---|
| AAV-F | TGCATGACCAGGCTCAGCTA |
| AAV-R | GACAGGGAAGGGAGCAGTG |
| Cas13a-F | GGAAAAGTACCAGTCCGCCA |
| Cas13a-R | GAAGTCCAGGAACTTGCCGA |
| GAPDH-F | ATTTGGTCGTATTGGGCG |
| GAPDH-R | CTCGCTCCTGGAAGATGG |
| Human RelA-F | CCTGGAGCAGGCTATCAGTC |
| Human Re A-R | ATGGGATGAGAAAGGACAGG |
| Mouse RelA-F | TGCGATTCCGCTATAAATGCG |
| Mouse RelA-R | ACAAGTTCATGTGGATGAGGC |
| Human GAPDH-F | ATTTGGTCGTATTGGGCG |
| Human GAPDH-R | CTCGCTCCTGGAAGATGG |
| Mouse GAPDH-F | TCACCACCATGGAGAAGGC |
| Mouse GAPDH-R | GCTAAGCAGTTGGTGGTGCA |
| Human FPN-F | CACAACCGCCAGAGAGGATG |
| Human FPN-R | CACATCCGATCTCCCCAAGT |
| Human Lcn2-F | CCCGCAAAAGATGTATGCCA |
| Human Lcn2-R | CTCACCACTCGGACGAGGTA |
| Mouse FPN-F | TGGAACTCTATGGAAACAGCCT |
| Mouse FPN-R | TGGCATTCTTATCCACCCAGT |
| Mouse Lcn2-F | TGGCCCTGAGTGTCATGTG |
| Mouse Lcn2-R | CTCTTGTAGCTCATAGATGGTGC |

1.13. Statistical Analysis

All data are presented as mean±standard error. Statistical analysis and graphing were performed using GraphPad Prism software (GraphPad Software). Data were statistically processed using analysis of variance and Student's t-test. Differences at p<0.05 were considered statistically significant. *, p<0.05; **, p<0.01; ***, p<0.001.

2. Results

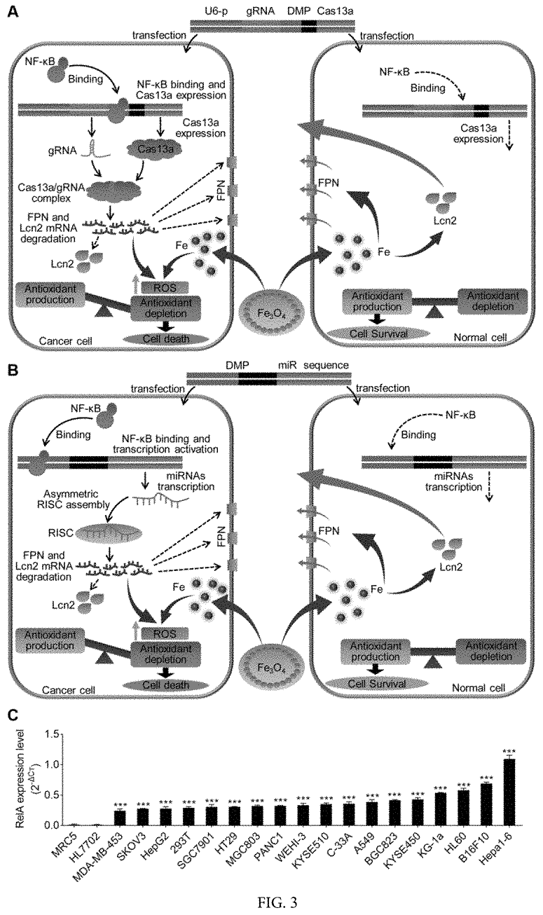

2.1. Principle and process of genetic interference ferroptosis therapy (GIFT) FIGS. 3A and 3B schematically illustrate the principle of gene interference ferroptosis therapy (GIFT). GIFT consists of a gene expression regulatory vector activated by transcription factor NF-κB and Fe₃O₄ nanoparticles (FeNPs). The NF-κB-activated gene expression regulatory vector consists of a promoter DMP and downstream effector genes, wherein the DMP promoter consists of a NF-κB decoy sequence and a minimal promoter sequence. DMP is a NF-κB specific promoter, and since NF-κB is a transcription factor that is overactivated in inflammation and cancer, DMP can be activated by NF-κB in NF-κB-overactivated cancer cells, which drives the expression of its downstream effector genes, while in normal cells without NF-κB expression, the DMP promoter cannot be activated, and its downstream effector genes are not expressed. Therefore, the DMP promoter is a cancer cell-specific activated promoter. When the DMP-controlled CRISPR/Cas13a or miRNA gene expression interference system is transfected into cancer cells, the overactivated NF-κB will bind to DMP to drive the expression of Cas13a or miRNA, and the expressed Cas13a protein can be activated with the U6 promoter. The expressed gRNA is assembled into a Cas13a/gRNA complex, and the miRNA is processed and bound to the RISC complex. Both Cas13a-gRNA and the miRNA-RISC complex can target and degrade the target mRNA, inhibiting or knocking down the expression of the target gene in cancer cells. In the present invention, two genes related to iron metabolism, namely FPN and Lcn2, are selected as target genes. The intracellular functions of FPN and Lcn2 are both related to the cellular efflux of iron. Therefore, by reducing the expression of these two genes in cancer cells, the active efflux of large amounts of iron ions produced by FeNPs can be prevented. The accumulation of iron ions leads to a significant increase in the level of intracellular ROS, which in turn leads to significant ferroptosis in cancer cells. In normal cells, the expression of Cas13a-gRNA or miRNA, the interference system of the two genes, cannot be produced, and its expression is not affected. The cells can actively efflux the iron ions generated after FeNPs enter the cells, and maintain iron homeostasis, thus not affecting normal cells nor making an impact.

2.2. Expression of NF-κB RelA in Cancer Cells and Normal Cells

NF-κB is widely activated in almost all types of tumor cells. Since the activity of intracellular NF-κB is crucial for the feasibility of the present invention, quantitative PCR was first used to detect three leukemia cells (KG-1a, HL60 and WEHI-3), other 15 cancer cells (including HEK-293T, HepG2, A549, HT-29, C-33A, SKOV3, PANC-1, MDA-MB-453, BGC-823/MGC-803/SGC-7901, KYSE450/KYSE510, Hepa1-6 and B16F10) and two Levels of NF-κB RelA/p65 in human normal cell lines (HL7702 and MRC5). The results showed that different levels of NF-κB RelA/p65 expression were detected in all cancer cell lines, but not in normal cell lines (MRC-5 and HL7702) (FIG. 3C). Therefore, the NF-κB-specific promoter DMP can be used to drive the specific expression of effector genes in cancer cells.

2.3. In Vitro Effects of FeNPs on Cells

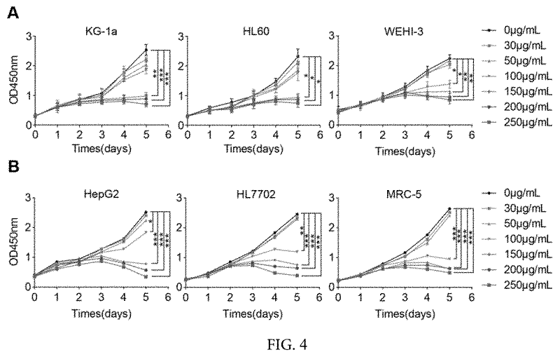

To evaluate the cytotoxic effects of FeNPs, three leukemia cells (including KG-1a, HL60 and WEHI-3), one solid tumor cell HepG2, and two human normal cells (HL7702 and MRC5) were incubated with various concentrations of FeNPs for five days, and at various time points after treatment, cell viability was analyzed using CCK-8, and cell growth curves were established. The results showed that FeNPs had no obvious cytotoxic effect on all six cell lines when the dose was lower than 50 g/mL during the incubation time (FIG. 4), but when the dose was greater than 50 g/mL, FeNPs had no obvious cytotoxic effect on normal cells. The growth of HL7702 and MRC-5 was significantly affected (FIG. 4B). Furthermore, cancer cells were more tolerant to FeNPs, and FeNPs treatment at 100 μg/mL had no significant effect on both human leukemia cells (KG-1a and HL60) (FIG. 4A), but not on mouse leukemia cells WEHI-3 and human hepatoma cells HepG2 have produced significant toxicity (FIG. 4B). Therefore, 50 μg/mL was used as a safe dose of FeNPs for further studies, which is equivalent to 3 mg kg-1 intravenously injected in rodents.

2.4. In Vitro Antitumor Effects of Gene-Interfering Ferroptosis Therapy (GIFT)

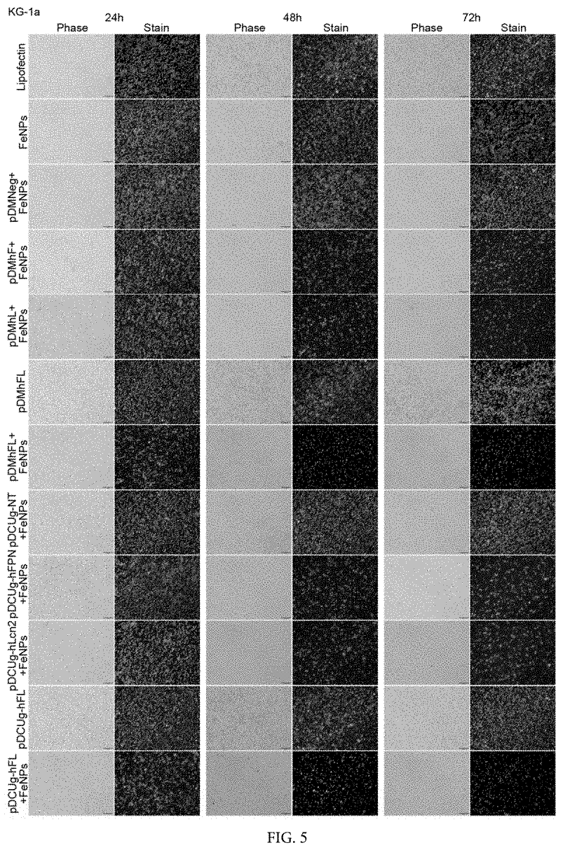

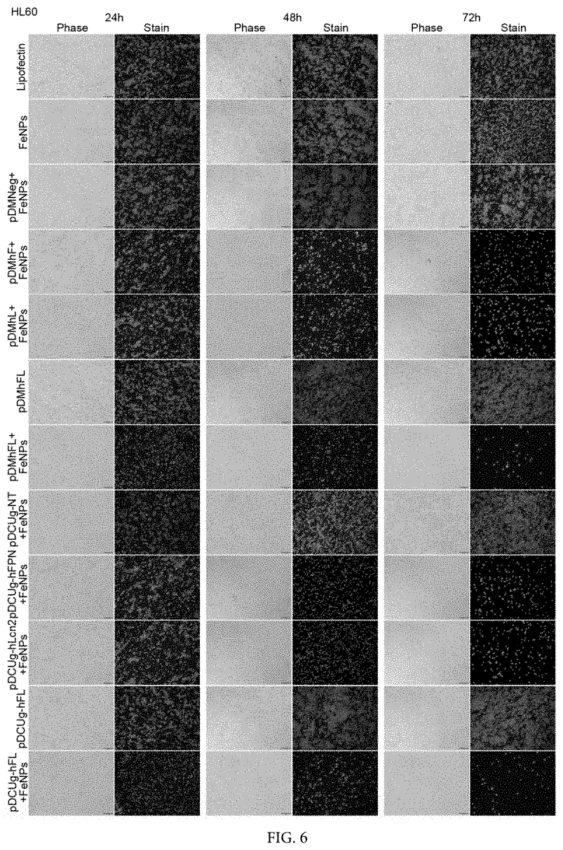

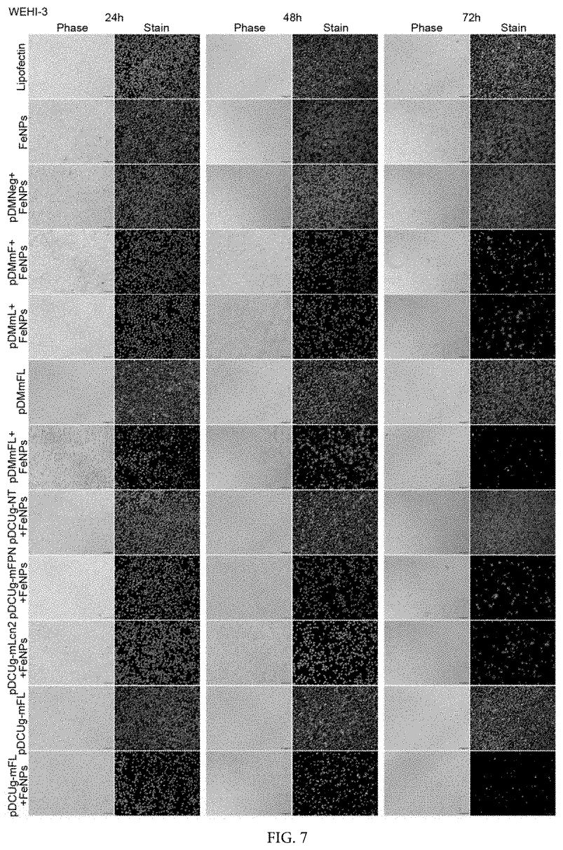

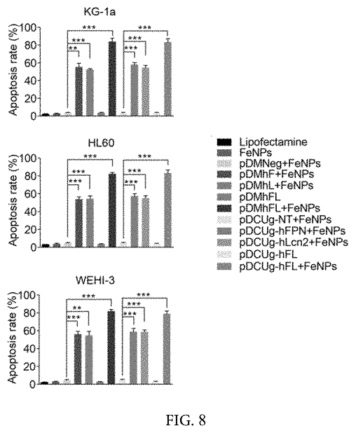

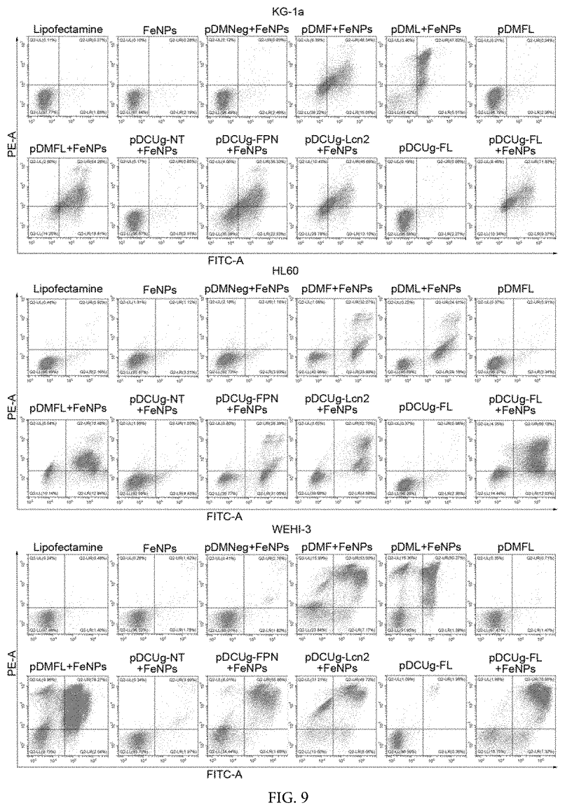

Firstly, the inhibitory effect of GIFT on leukemia cells was investigated. Use various plasmid vectors including pDCUg-NT, pDCUg-hFPN/pDCUg-mFPN, pDCUg-hLcn2/pDCUg-mLcn2, pDCUg-hFL/pDCUg-mFL, pDMNeg, pDMhF/pDMmF, pDMhL/pDMmL and pDMhFL/pDMmFL vectors, were transfected with three kinds of leukemia cells (KG-1a, HL60 and WEHI-3) in the 24-well plate respectively. Two-four hours after transfection, the cells were cultured with the culture medium containing live cells but not containing 50 μg/mL FeNPs for 24 hours, 48 hours and 72 hours respectively, and the cells were detected by acridine orange/ethidium bromide double staining. Dead and alive, and cells treated in parallel were collected at the 72-hour time point for quantitative detection of apoptosis. The results showed that by combining with the vectors pDCUg-hFPN/pDCUg-mFPN, pDCUg-hLcn2/pDCUg-mLcn2, pDCUg-hFL/pDCUg-mFL, pDMhF/pDMmF, pDMhL/pDMmL and pDMhFL/pDMmFL, FeNPs causes three leukemia cells significant apoptosis in all of them (FIG. 5, FIG. 6, FIG. 7); and this anticancerous effect was obviously time-dependent (FIG. 5, FIG. 6, FIG. 7). However, each plasmid alone, FeNPs, and the combination of negative plasmids (pDCUg-NT and pDMNeg) with FeNPs did not significantly affect all cells at any treatment time (FIG. 5, FIG. 6, FIG. 7). More importantly, FeNPs produced the strongest cancer cell killing effect when two gene interference vectors (pDCUg-hFL/pDCUg-mFL and pDMhFL/pDMmFL) were co-expressed (FIG. 5, FIG. 6, FIG. 7), a synergistic effect of the co-interference of the two genes was shown. In addition, the quantitative detection of apoptosis of cells with lost cells also showed the same results as the detection of acridine orange/ethidium bromide double staining (FIG. 8, FIG. 9).

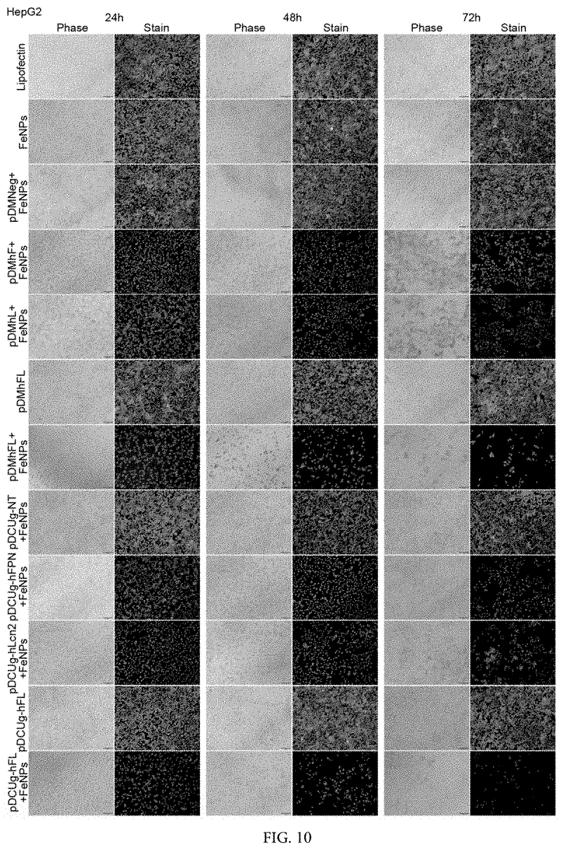

Then, the inhibitory effect of GIFT on solid tumor cells was examined. Human hepatoma cells HepG2 were transfected in 24-well plates with various plasmid vectors including pDCUg-NT, pDCUg-hFPN, pDCUg-hLcn2, pDCUg-hFL, pDMNeg, pDMhF, pDMhL and pDMhFL vectors, respectively. 24 hours after transfection, the cells were cultured with a culture medium containing viable but not containing 50 μg/mL FeNPs for 24 hours, 48 hours and 72 hours, respectively, and the cell death was detected by acridine orange/ethidium bromide double staining, and at 72 hours. Cells treated in parallel were collected at various time points, and apoptosis was quantified. The results showed that FeNPs caused significant apoptosis of HepG2 cells by combining with the vectors pDCUg-hFPN, pDCUg-hLcn2, pDCUg-hFL, pDMhF, pDMhL and pDMhFL (FIG. 10); and this anticancerous effect also showed obvious time dependence (FIG. 10). However, each plasmid alone, FeNPs, and the combination of negative plasmids (pDCUg-NT and pDMNeg) with FeNPs did not have a significant effect on HepG2 cells at any treatment time (FIG. 10). Similarly, when two gene interference vectors (pDCUg-hFL and pDMhFL) were co-expressed, FeNPs produced the strongest cancer cell killing effect (FIG. 10), also showing the synergistic effect of the two gene co-interference.

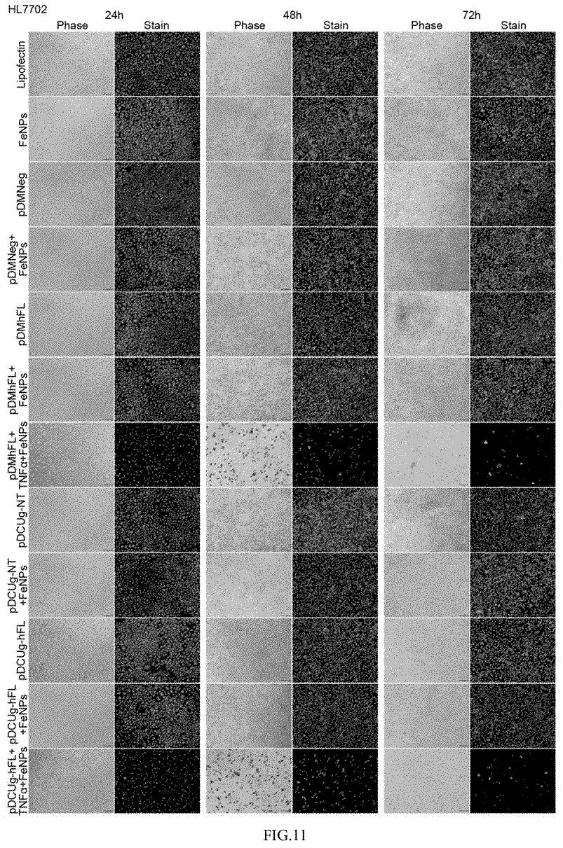

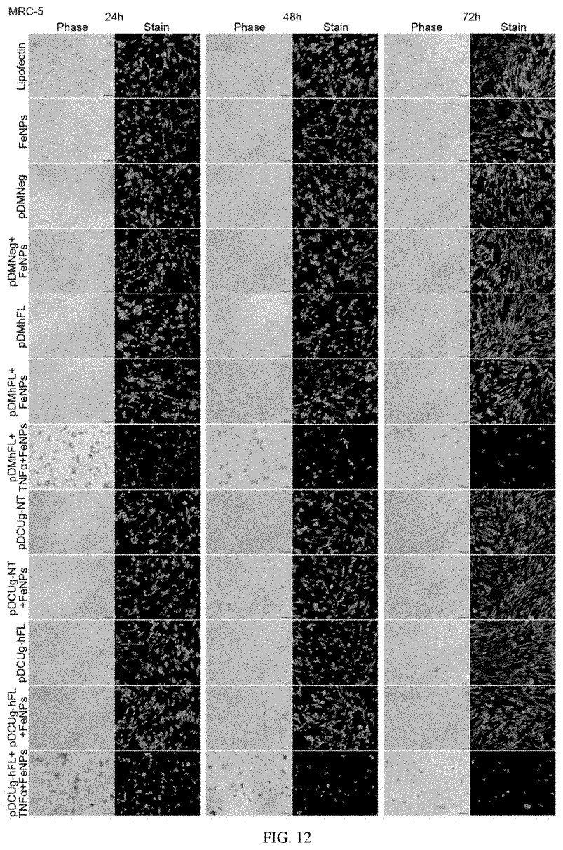

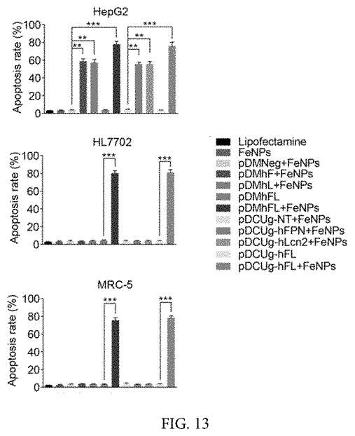

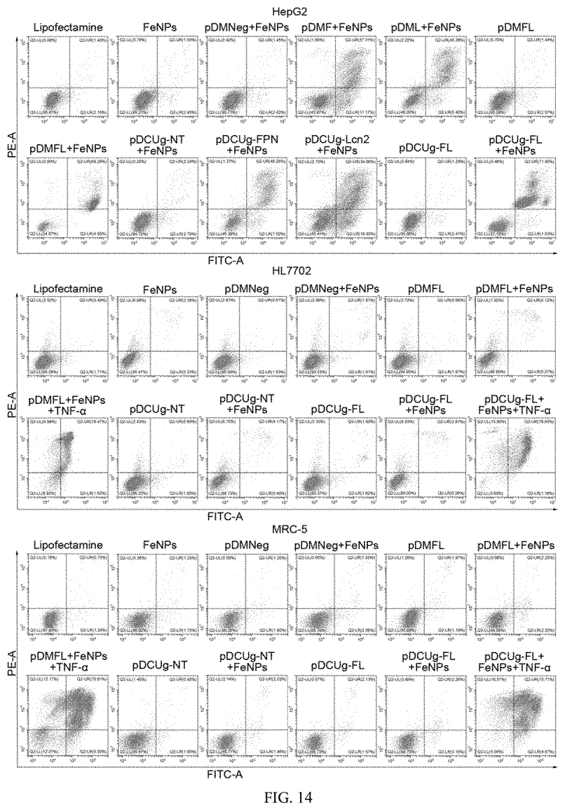

To examine the cancer cell specificity of GIFT, two human normal cells, HL7702 and MRC5, were treated in the same manner as HepG2. The results showed that none of the vectors, alone or in combination with FeNPs, had a significant effect on either (FIG. 11, FIG. 12), which is consistent with the result that NF-κB expression was not detected in these two normal cells (FIG. 3C). To further observe the essential role of NF-κB activation on GIFT, these two cells were firstly transfected with pDCUg-hFL and pDMhFL, and then treated with the NF-κB activator TNF-α, followed by FeNPs. It was found that these two normal cells were also significantly killed by GIFT (FIG. 11, FIG. 12). It shows that only NF-κB activation, GIFT can play a role in killing cancer cells. In addition, the quantitative measurement of cell apoptosis with the loss of cells also showed the same results as the acridine orange/ethidium bromide double staining detection (FIG. 13, FIG. 14).

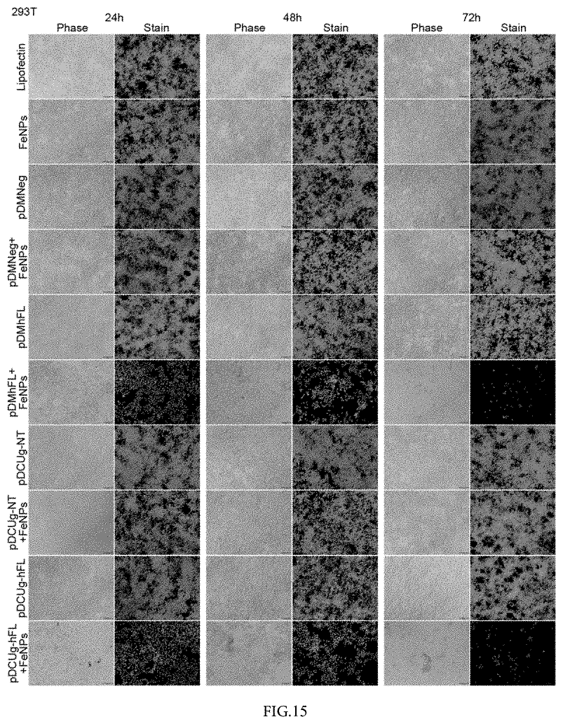

Only NF-κB activation of GIFT was also observed in HEK-293T cells. HEK-293T cells are human embryonic kidney cells transfected with a virus that expresses the large T antigen. Although these cells are not considered to be cancer cells, their NF-κB expression is significantly activated (FIG. 3C). Therefore, the combination of pDCUg-hFL and pDMhFL vectors with FeNPs also produced a significant killing effect on this cell (FIG. 15).

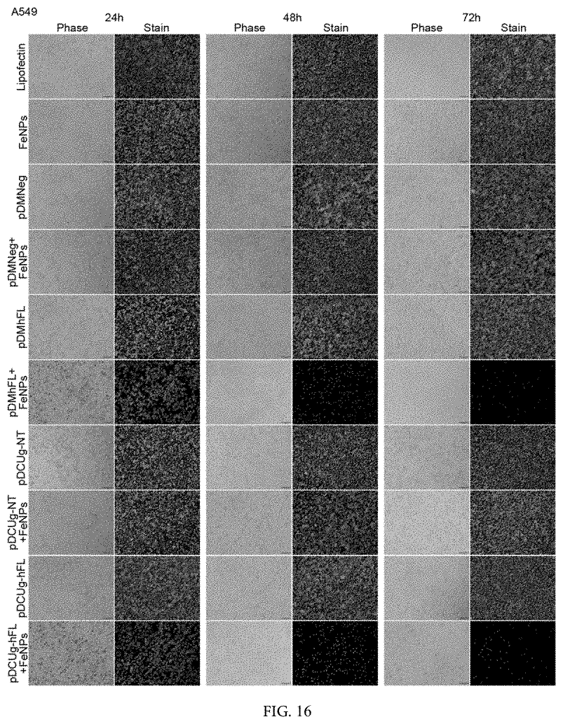

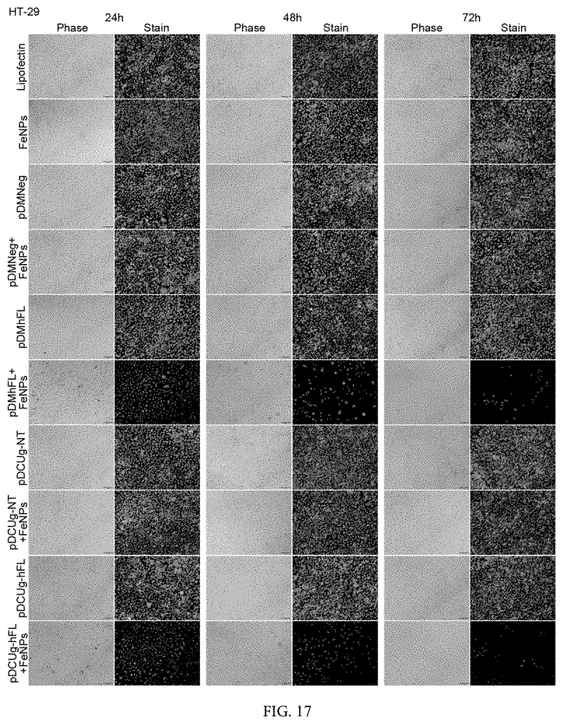

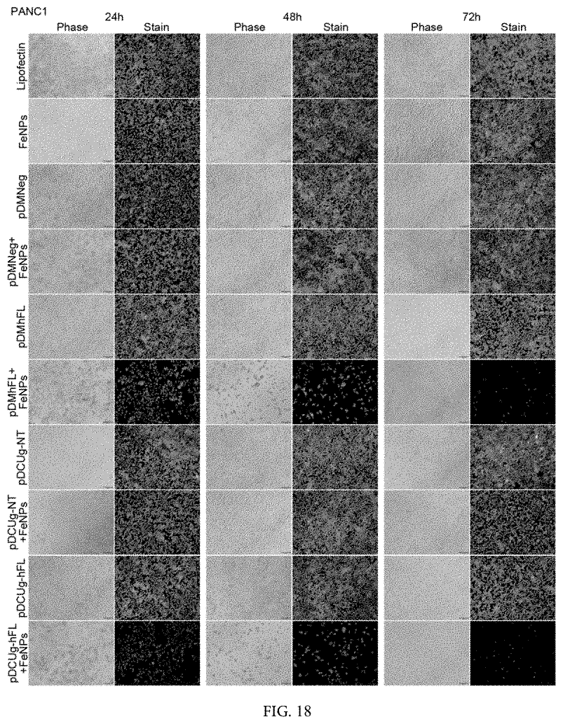

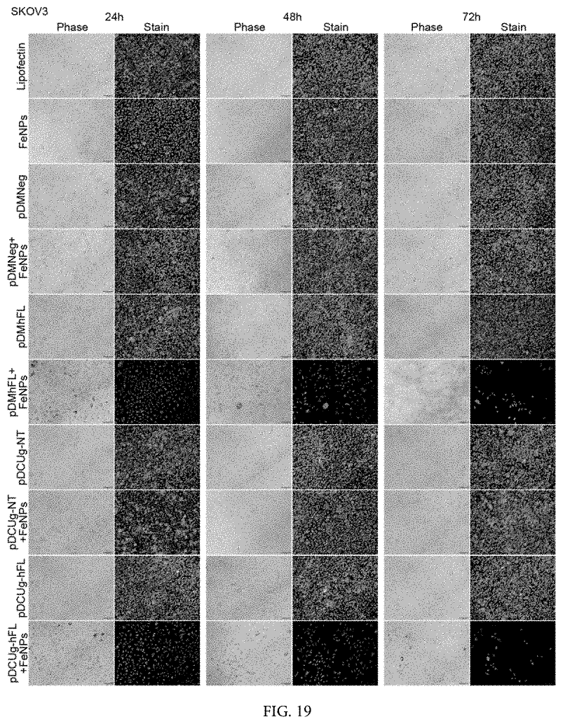

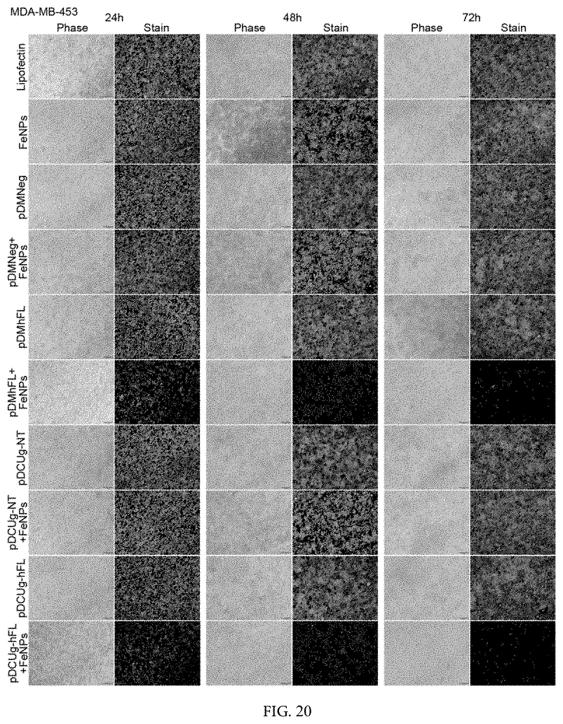

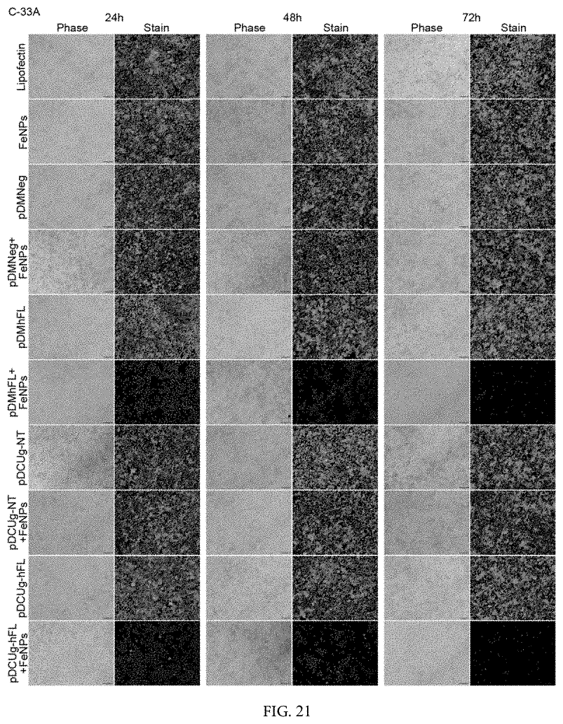

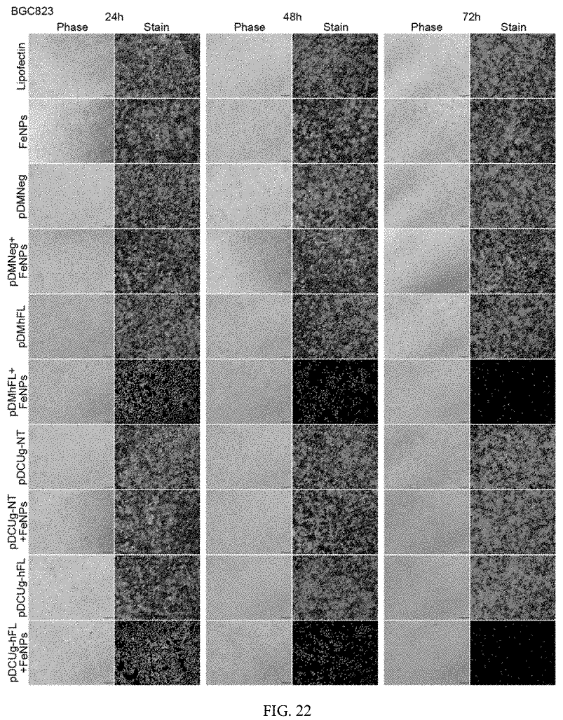

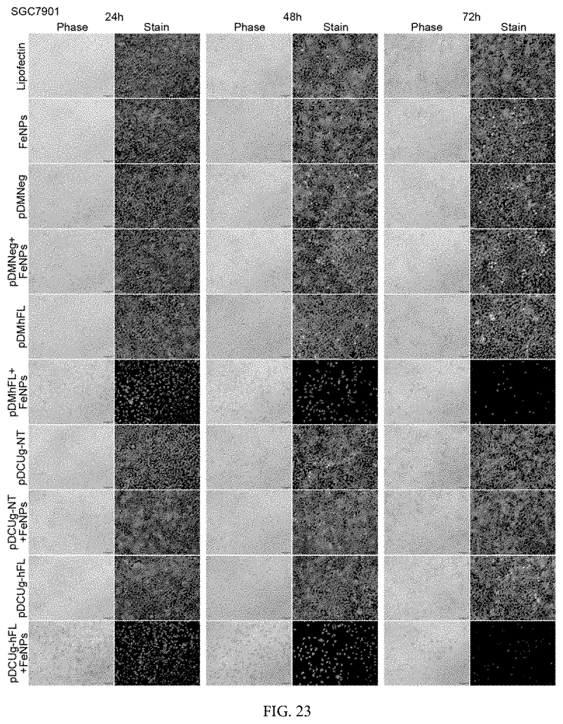

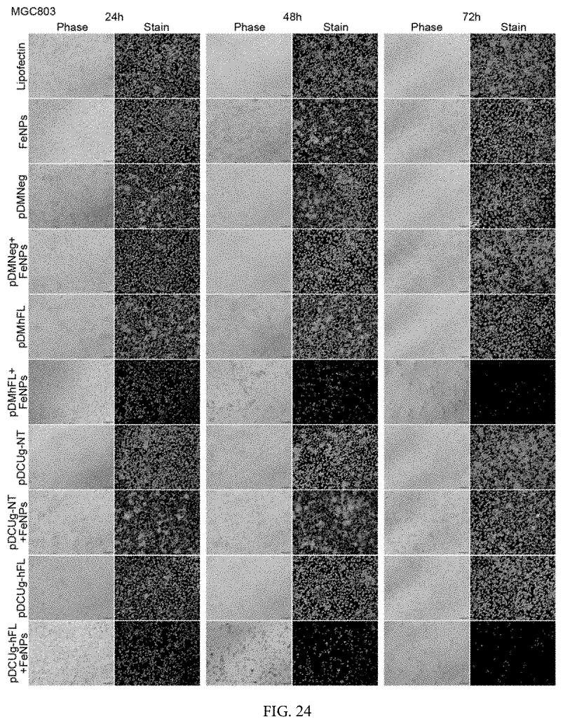

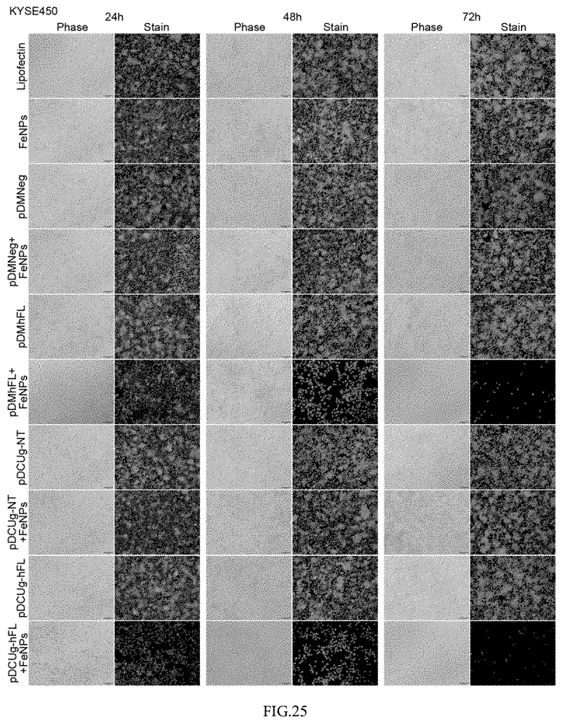

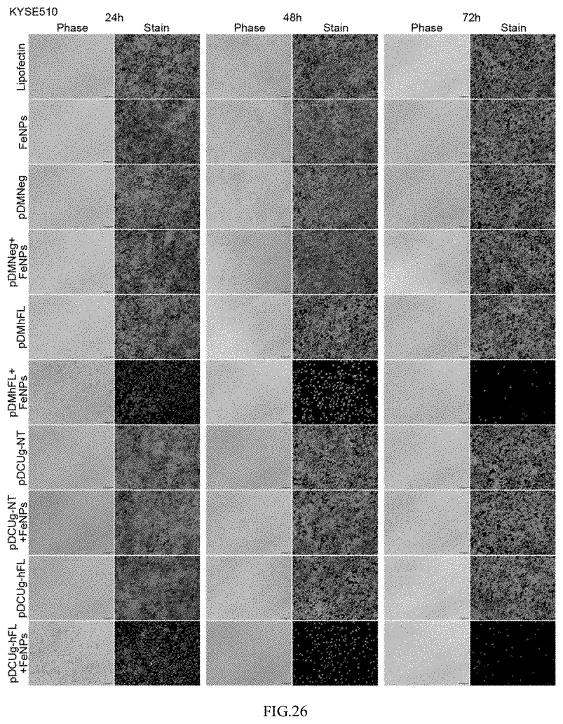

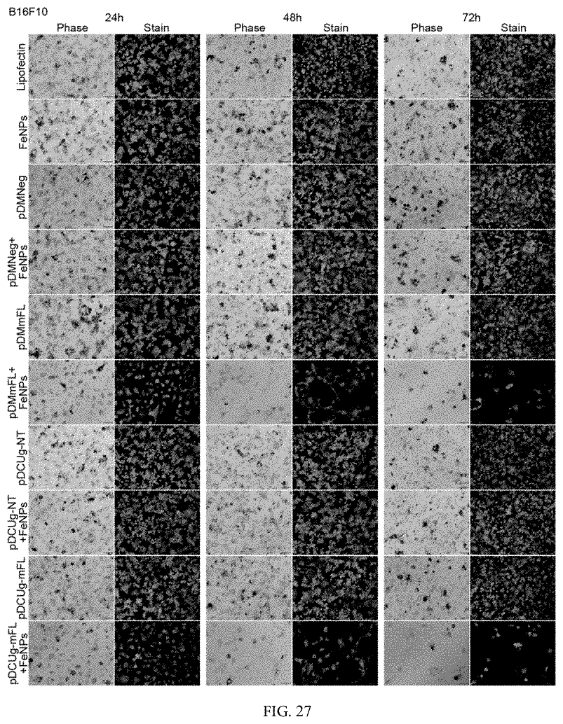

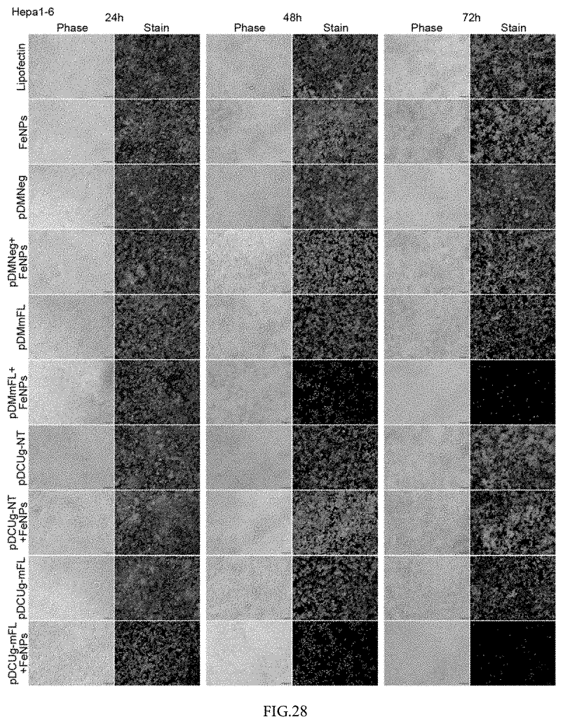

To investigate whether the GIFT mechanism has broad-spectrum anti-cancerous effect, a variety of cancer cells representing different human and mouse tumors, including A549, HT-29, C-33A, SKOV3, PANC-1, MDA-MB-453, BGC-823/MGC-803/SGC-7901, KYSE450/KYSE510, Hepa1-6, B16F10, were treated with the same treatment method. Since co-expression of the vectors produced the most significant cancer cell killing effects in the three leukemia cells and human hepatoma cell HepG2 cell experiments, only the pDCUg-hFL/pDCUg-mFL and pDMhFL/pDMmFL vectors were used in experiments with more cancer cells. The results showed that when the pDCUg-hFL/pDCUg-mFL and pDMhFL/pDMmFL vectors were combined with FeNPs, they had a significant anti-cancerous effect on various cancer cells (FIG. 16˜FIG. 28); and this anti-cancerous effect was also critically time-dependent (FIG. 16-FIG. 28). Likewise, each vector alone, FeNPs, and the combination of negative plasmids (pDCUg-NT and pDMNeg) with FeNPs did not significantly affect all cells at any treatment time (FIGS. 16-28).

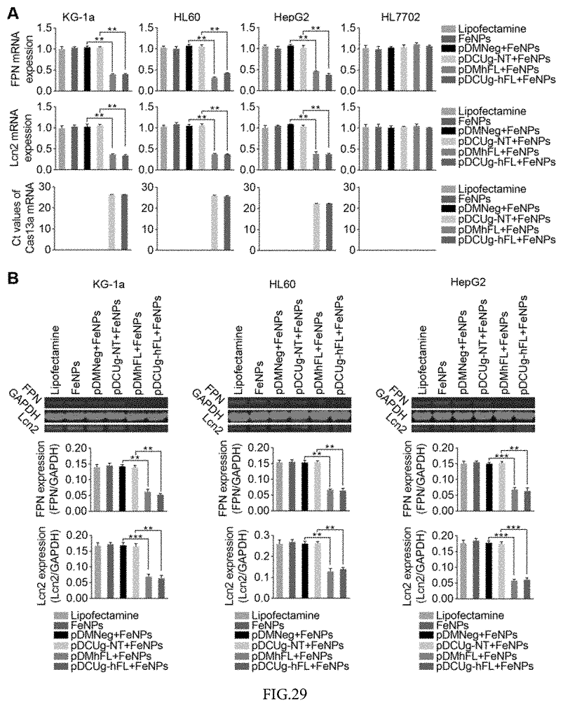

To evaluate the knockdown effect of two tools, DMP-Cas13a-U6-gRNA (pDCUg) and DMP-miR (pDMP-miR), the expression levels of FPN and Lcn2 genes were detected by qPCR. The results showed that targeting gRNA/miRNA significantly down-regulated the level of target mRNA in cancer cells KG-1a, HL60 and HepG2 cells (FIG. 29A). However, no changes were found in normal cells HL7702 cells, further indicating the NF-κB specificity of the DMP promoter as well as the cancer cell specificity (i.e., only works in cancer cells). To further explore the specific expression of effector genes in cancer cells, the expression levels of Cas13a mRNA under various treatments were detected in the above four cells. The results showed that Cas13a was only expressed in cancer cells KG-1a, HL60 and HepG2 transfected with pDCUg-hFL and pDMhFL (FIG. 29A); however, Cas13a mRNA was not detected in normal cells HL7702 under all treatments (FIG. 29A). These results suggest that the DMP-based gene expression system can be activated in cancer cells but not normal cells, which leads to the specific expression of effector genes only in cancer cells. Afterwards, the protein levels of FPN and Lcn2 were detected by western blotting (WB). The results showed that the expression of FPN and Lcn2 proteins was significantly inhibited in cancer cells KG-1a, HL60 and HepG2 transfected with targeting plasmids (pDMhFL and pDCUg-hFL) (FIG. 29B).

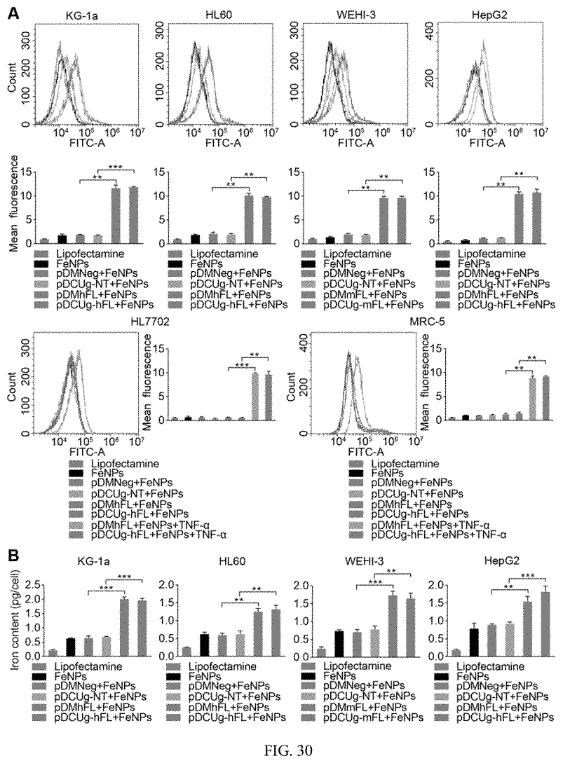

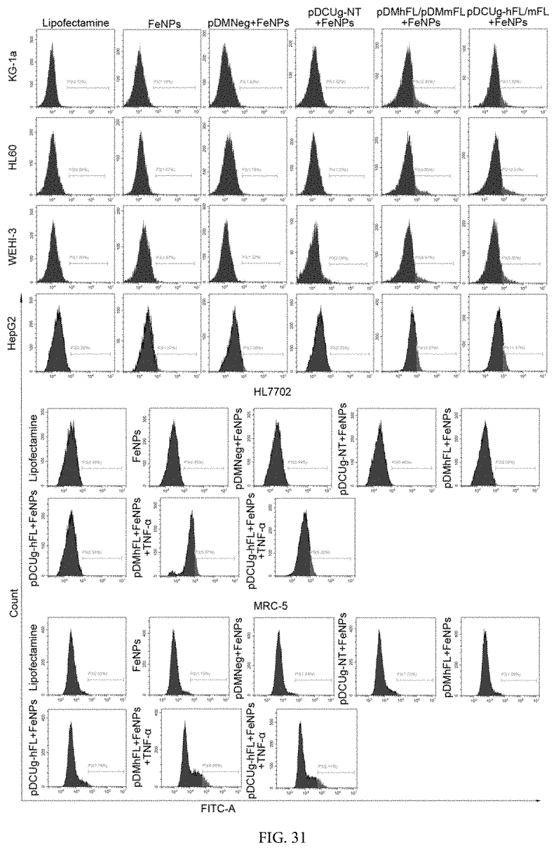

Iron-based nanomaterials can upregulate ROS levels through the Fenton reaction, resulting in specific killing effects in cancer. In order to investigate whether the Fenton reaction occurs in the co-cultures of the present invention and to explore the underlying mechanism of GIFT-induced apoptosis in cancer cells, we measured the effect of GIFT in three leukemia KG-1a, HL60 and WEHI-3 and one solid tumor cell HepG2 in Intracellular active ROS levels and intracellular iron content under treatment of various plasmids (pDCUg-hFL, pDCUg-NT, pDMhFL and pDMNeg) in combination with FeNPs. The results showed that three leukemia cells and HepG2 had increased intracellular ROS levels in all treatments containing FeNPs (FIG. 30A; FIG. 31), especially when treated with pDCUg-hFL and pDMhFL vectors in combination with FeNPs. ROS levels in cancer cells were dramatically elevated (FIG. 30A; FIG. 31), which is consistent with the significant apoptosis of these cancer cells under the same treatments indicated by the above assays (FIG. 8; FIG. 13). Intracellular iron content assays showed that the four cancer cells had increased intracellular iron content in all treatments containing FeNPs (FIG. 30B), especially when treated with pDCUg-hFL and pDMhFL vectors in combination with FeNPs. The iron content in the cells increased dramatically (FIG. 30B), which is consistent with the dramatic increase in ROS levels in the four cancer cells under the same treatment indicated by the above assays. It shows that the inhibition of the expression of iron efflux-related target genes FPN and Lcn2 in cancer cells combined with FeNPs can cause a sharp increase in the intracellular iron content, thereby triggering a sharp increase in the level of intracellular ROS, and ultimately leading to massive apoptosis of cancer cells. This indicates that the mechanism of GIFT killing cancer cells is the ferroptosis amplified by the gene interference designed by the present invention.

2.5. Antitumor Effect of GIFT Based on Viral Vectors In Vitro (Evaluation of Viral Vectors)

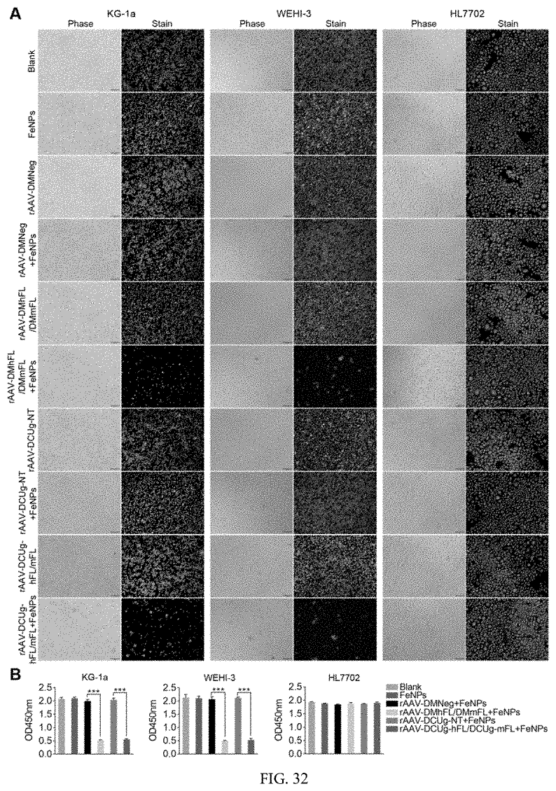

To determine whether the combination of FeNPs with pDMP-Cas13a-U6-gRNA or pDMP-miRNA affects tumor growth in vivo. DMP-Cas13a-U6-gRNA and DMP-miRNA were packaged into AAV vectors to construct recombinant viruses rAAV-DCUg-NT, rAAV-DCUg-Hfl/rAAV-DCUg-mFL, rAAV-DMNeg and rAAV-DMhFL/rAAV-DMmFL. Three cells, KG-1a, WEHI-3 and HL7702, were infected with the recombinant viruses, after which the cells were incubated with or without FeNPs for an additional 72 hours. The results showed that combined treatment of two targeted rAAVs (rAAV-DCUg-hFL/mFL and rAAV-DMhFL/DMmFL) with FeNPs, compared to two non-targeted rAAVs (rAAV-DCUg-NT and rAAV-DMNeg), Caused significant apoptosis of KG-1a and WEHI-3 cells. Whereas rAAV-DCUg-NT and rAAV-DMNeg viruses alone, FeNPs, and the combination of rAAV-DCUg-NT and rAAV-DMNeg viruses with FeNPs did not significantly affect the growth of these two leukemia cells (FIG. 32). In HL7702 cells, treatment of all viruses with FeNPs alone or in combination did not cause significant apoptosis (FIG. 32).

2.6. Iron Nanocarriers (FeNCs)-Based GIFT Inhibits Cancer Cells (Evaluation of Nanosiderophores)

In order to explore whether iron nanoparticles can be used as gene carriers, the gene carrier and iron nanoparticles were combined into one, as a reagent to achieve GIFT treatment, a PEI-modified Fe₃O₄ (FeNCs) was selected as the DNA transfection agent, and the It is defined as iron nanocarriers (FeNCs). Two GIFT inhibition experiments on cancer cells were performed using two batches of FeNCs (FeNCs-1 and FeNCs-2).

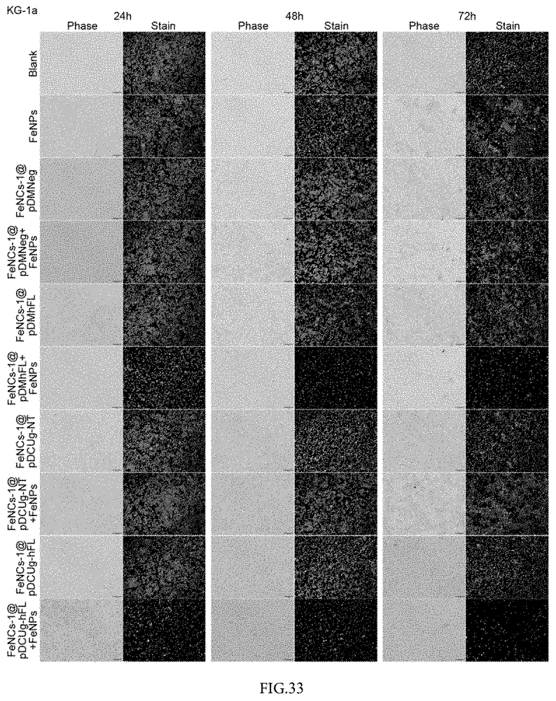

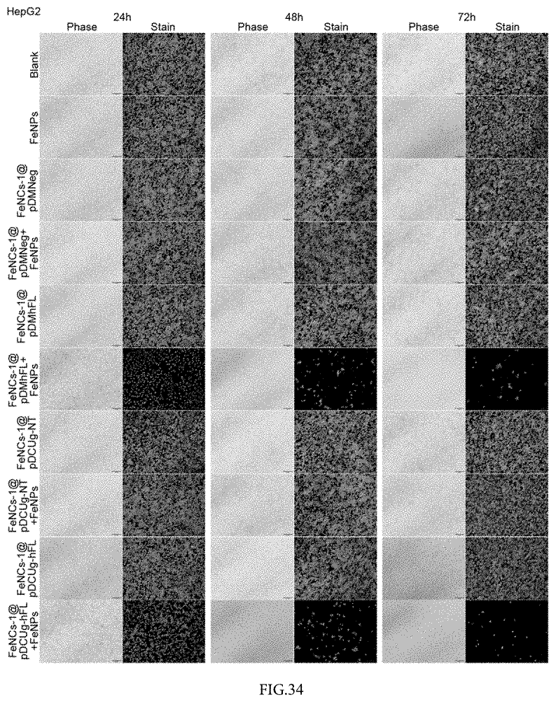

In the first GIFT inhibition experiment based on FeNCs, four plasmids (pDCUg-NT, pDCUg-hFL, pDMNeg and pDMhFL) were loaded with FeNCs-1 to prepare FeNCs loaded with plasmid DNA (FeNCs@DNA) to obtain FeNCs −1@pDCUg-NT, FeNCs-1@pDCUg-hFL, FeNCs-1@pDMNeg and FeNCs-1@pDMhFL. Blood cancer cells KG-1a were first treated with these FeNCs-1@DNA for DNA transfection, and then the cells were retreated with 50 μg/mL FeNPs, and cell growth was detected by acridine orange/ethidium bromide staining at different time points. The results showed that FeNPs alone and FeNCs@DNA treatment of cells did not significantly affect cell growth (FIG. 33); however, when cells were co-treated with FeNCs-1@pDCUg-hFL and FeNCs-1@pDMhFL with FeNPs, cells Significant time-dependent death occurred (FIG. 33), while co-treatment of cells with FeNCs-1@pDCUg-NT and FeNCs-1@pDMNeg with FeNPs did not have a significant effect on cell growth (FIG. 33). Solid tumor cells HepG2 were also treated using the same method with similar results (FIG. 34). It shows that the use of iron nanoparticles as a gene transfection agent can also introduce gene interference vectors into cells, resulting in the effect of GIFT on inhibiting cancer cells. In addition, this experiment also showed that although FeNCs-1 is also nano-iron, due to the limited dose used, treating both cancer cells with FeNCs-1@pDCUg-hFL and FeNCs-1@pDMhFL alone did not affect their growth.

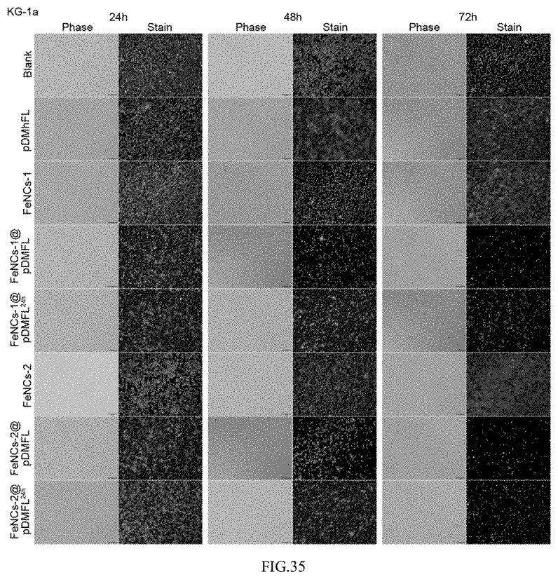

Obviously, it is cumbersome to use two kinds of bulk nanoparticles (FeNPs and FeNCs). Therefore, in the second FeNCs-based GIFT inhibition experiment on cancer cells, two plasmids (pDMNeg and pDMhFL) were mixed with FeNCs-1 and FeNCs-2, respectively, to prepare FeNCs@DNA to obtain FeNCs-1@pDMhFL and FeNCs-2@pDMhFL. The leukemia cells KG-1a were treated with the prepared FeNCs-1@pDMhFL and FeNCs-2@pDMhFL at a dose of 50 μg/mL. The results showed that neither FeNCs nor DNA treatment of cells had a significant effect on cell growth (FIG. 35); however, when cells were treated with FeNCs-1@pDMhFL and FeNCs-2@pDMhFL, cells experienced significant time-dependent death (FIG. 35). In order to further investigate the stability of FeNCs@DNA, that is, whether DNA would fall off from FeNCs in a short time, affecting the efficiency of transfected cells in vivo, the prepared FeNCs-1@pDMhFL and FeNCs-2@pDMhFL were placed for 24 hours (FeNCs-1@pDMhFL). @DNA has a certain time to reach cancer cells after intravenous injection), and then it is used to treat cells. The results showed that FeNCs@DNA had a similar killing effect on cancer cells after placement (FIG. 35).

2.7. In Vivo Antitumor Effect of GIFT

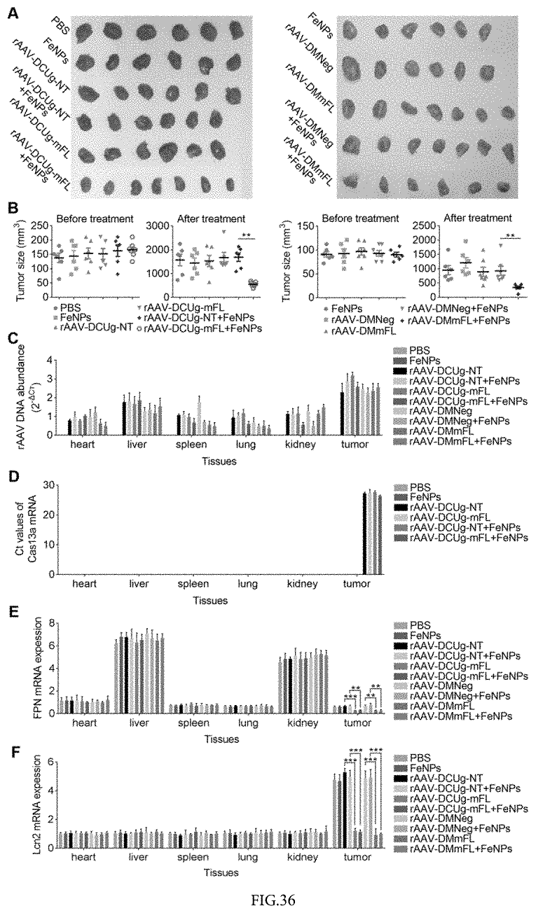

Animal experiments: WEHI-3 cells were transplanted to BALB/c female mice, and three batches of animal experiments were carried out. Tumor inhibition experiments based on rAAV-DCUg-mFL were carried out in the second batch of animal experiments. Six groups of tumor-bearing mice were treated with different treatments, including PBS, FeNPs, rAAV-DCUg-NT, rAAV-DCUg-NT+FeNPs, rAAV-DCUg-mFL, and rAAV-DCUg-mFL+FeNPs. The results showed that the rAAV-DCUg-mFL+FeNPs treatment group produced a significant tumor-inhibiting effect, while the other treatment groups did not produce a significant tumor-inhibiting effect (FIG. 36A and FIG. 36B). Tumor inhibition experiments based on rAAV-DMmFL were carried out in the second batch of animal experiments. Five groups of tumor-bearing mice were treated with different treatments, including FeNP, rAAV-DMNeg, rAAV-DMmFL, rAAV-DMNeg+FeNPs, and rAAV-DMmFL+FeNPs. The results showed that the rAAV-DMmFL+FeNPs treatment group had a significant tumor-suppressing effect, while the other treatment groups had no significant tumor-suppressing effect (FIG. 36A and FIG. 36B).

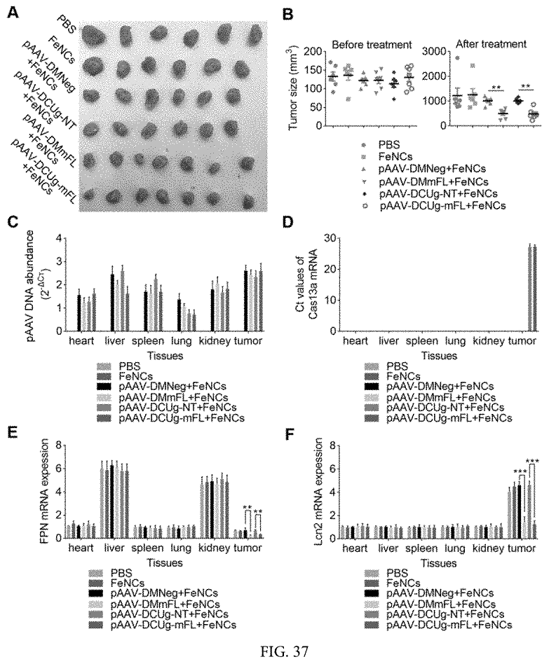

In the third batch of animal experiments, tumor inhibition experiments based on iron nanoparticles directly loaded with plasmid DNA were investigated. In this experiment, FeNCs, a DNA transfection reagent based on iron oxide nanomaterials, were used for in vivo DNA delivery. Six groups of tumor-bearing mice were treated with different treatments, including PBS, FeNCs, pAAV-DMNeg+FeNCs, pAAV-DMmFL+FeNCs, pAAV-DCUg-NT+FeNCs, and pAAV-DCUg-mFL+FeNCs. The results showed that FeNCs loaded with two targeting plasmids (pAAV-DCUg-mFL and pAAV-DMmFL) significantly inhibited tumor growth, while FeNCs alone were significantly inhibited with two non-targeting plasmids (pAAV-DCUg-NT), and pAAV-DMNeg) FeNCs did not produce significant tumor growth inhibition (FIG. 37A and FIG. 37B).

To further demonstrate the tumor-specific expression of rAAV vectors in vivo, the abundance of rAAV DNA (first and second animal experiments) and pAAV DNA (third batch of animal experiments) in various tissues in three batches of animal experiments was detected, and the expression of Cas13a and target genes. qPCR detection showed that rAAV DNA and pAAV DNA were distributed to different degrees in various tissues, but the highest distribution level was in tumor tissue, followed by liver (FIG. 36C, FIG. 37C). qPCR detection also showed that Cas13a mRNA only appeared in tumor tissue, that is, Cas13a was only expressed in tumor tissue (FIG. 36D, FIG. 37D); in addition, the two target genes FPN and Lcn2 were expressed to different degrees in various tissues. The expression of FPN gene was highest in liver and kidney tissues, while the expression of Lcn2 gene was highest in tumor tissues (FIGS. 36E and 36F, FIG. 37E and FIG. 37F). After various treatments, the expression of these two target genes was significantly down-regulated only in tumors by treatments containing rAAV-DCUg-mFL, rAAV-DMmFL, pAAV-DCUg-mFL, and pAAV-DMmFL (FIGS. 37E and 37F). These results indicate that Cas13a and miRNA controlled by DMP are activated and expressed only in tumor tissues in vivo, thereby knocking down the expression of target genes only in tumor tissues, reflecting the tumor-specific activation of DMP promoter in vivo.

2.8. GIFT Targeting Other Genes Inhibits Cancer Cell Growth In Vitro

In order to further investigate whether GIFT targeting other genes also has similar anti-tumor effects, and to further the mechanism of GIFT killing tumor cells, pDMP-miR vectors targeting other five genes were designed and constructed, namely FSP1, FTH1, GPX4, NRF2 and SLC7A11, and miRNAs targeting two targets were designed for each gene. The constructed vectors were named pDMhFSP1-1, pDMhFSP1-2, pDMhFTH1-1, pDMhFTH1-2, pDMhGPX4-1, pDMhGPX4-2, pDMhNRF2-1, pDMhNRF2-2, pDMhSLC7A11-1 and pDMhSLC7A11-2, respectively. The selected five genes are closely related to cellular iron metabolism, ROS regulation and ferroptosis. Among them, GPX4 and FSP1 are ferroptosis-related genes, FTH1 is a ferritin-encoding gene involved in intracellular iron storage, and NRF2 is a redox-related transcription factor, SLC7A11, is a cystine membrane import protein involved in the synthesis of the intracellular reducing agent glutathione. FTH1 helps to store excess iron ions in cells to maintain intracellular iron homeostasis; SLC7A11 imports cystine into cells so that cells can synthesize glutathione to remove intracellular ROS; it is speculated to use pDMP targeting these genes Knockdown of their expression in cancer cells by -miR vector is beneficial to increase the intracellular iron ion content and increase the ROS level when FeNPs treat cells, which is beneficial to promote ferroptosis.

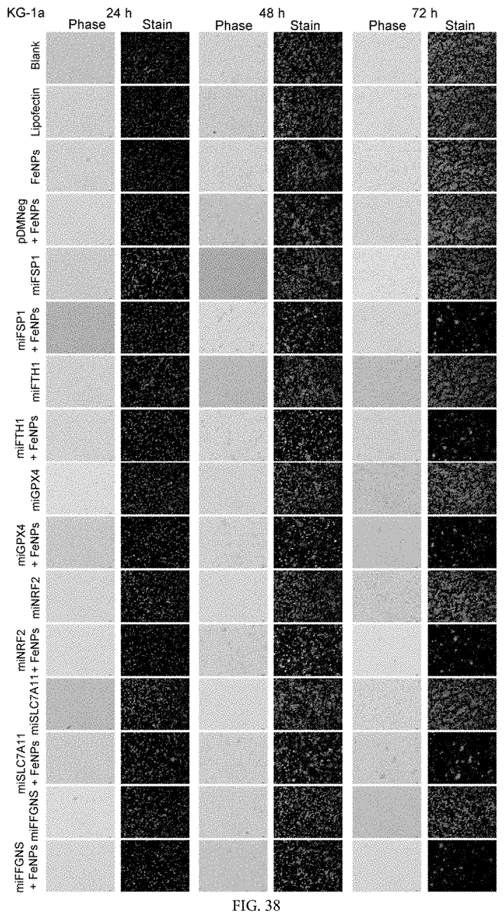

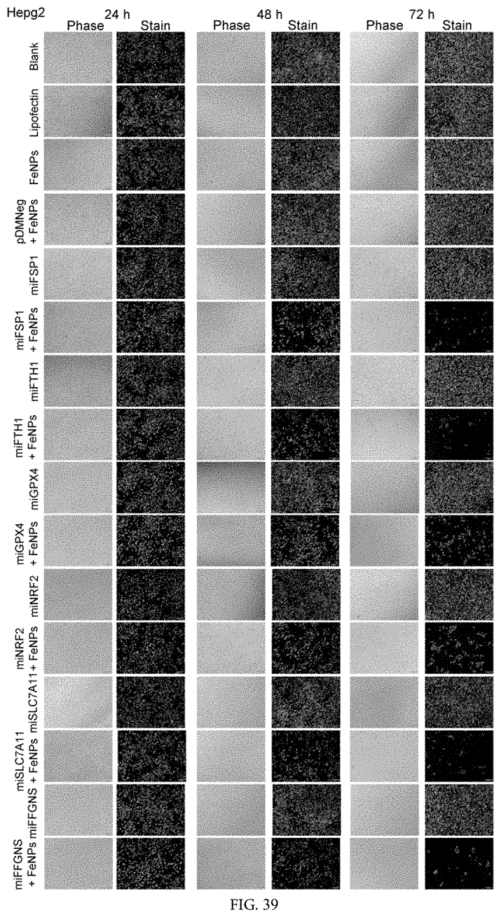

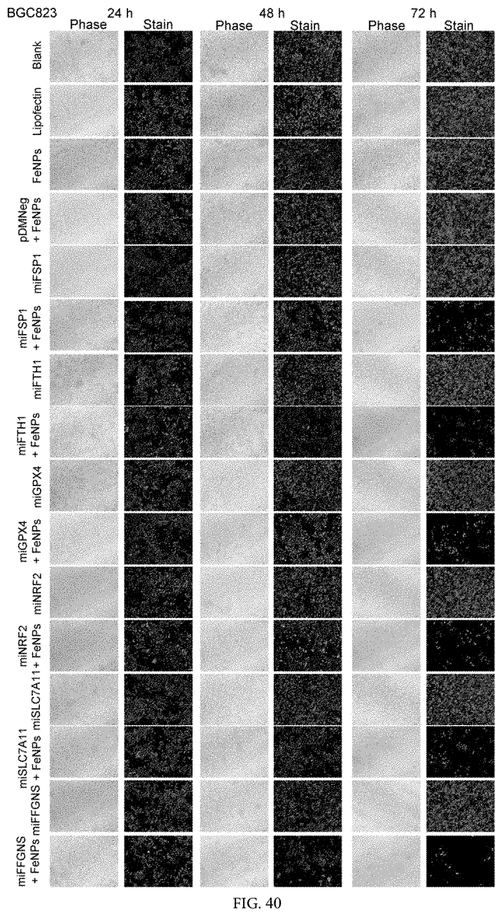

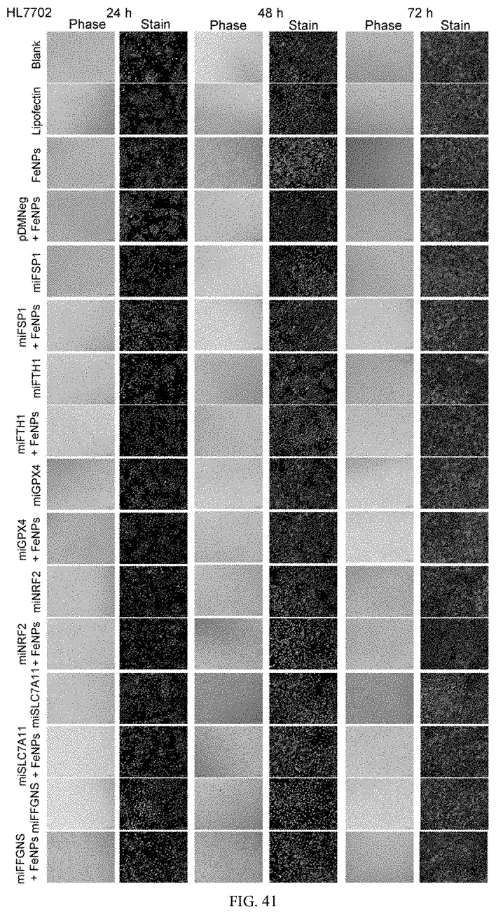

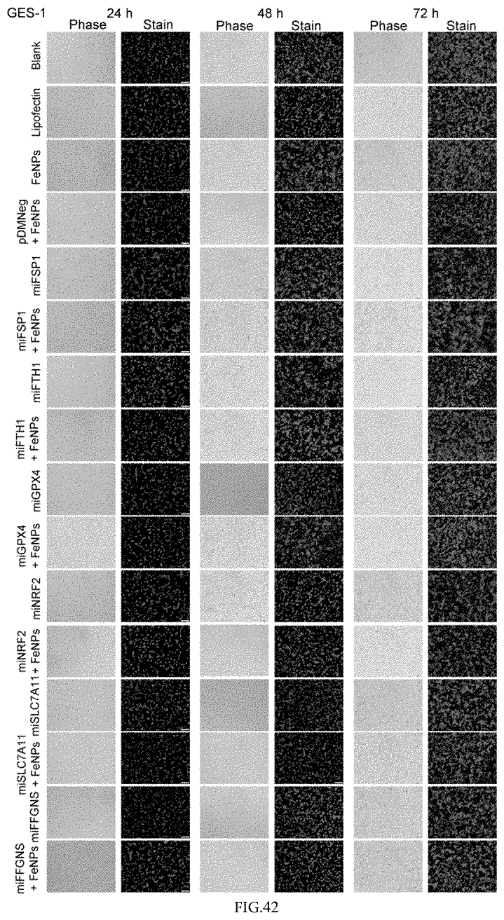

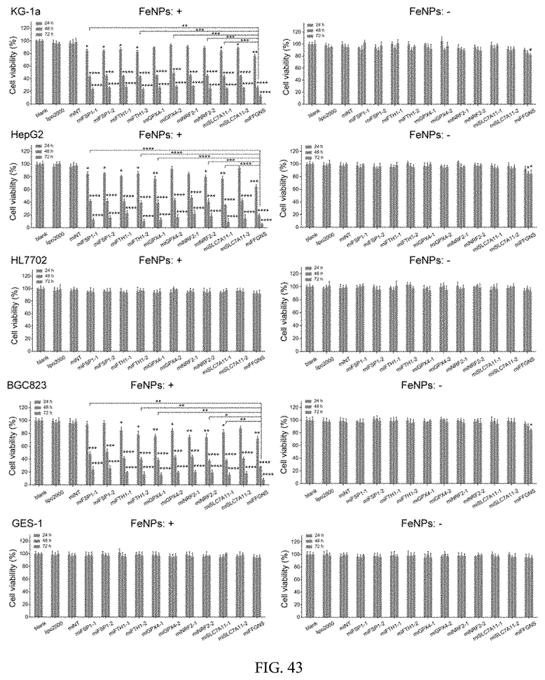

By selecting one leukemia cell KG-1a, two solid tumor cells HepG2 (human hepatoma cells) and BGC823 (human gastric cancer cells), and two corresponding human normal cells HL7702 (human normal hepatocytes) and GES-1 (human normal gastric mucosal epithelial cells) to test the above vectors. The cell viability was measured by acridine orange/ethidium bromide staining and CCK-8 method of cells treated at different time points. The results showed that each carrier alone had no significant effect on the growth of the above five cells (FIG. 38-FIG. 42).), and when they were used in combination with FeNPs, they produced significant time-dependent killing effects on all 3 cancer cells (KG-1a, HepG2, BGC823) (FIG. 38-FIG. 40), but not on both normal cells significant effect (FIG. 41, FIG. 42). The negative control vector pDMNeg alone or in combination with FeNPs had no significant effect on the growth of the above five types of cells (FIG. 38-FIG. 42). In addition, co-transfection (miFFGNS) experiments of 5 gene pDM vectors were also performed, and it was found that this co-transfection could significantly inhibit 3 types of cancer cells (KG-1a, HepG2, BGC823) even in the absence of FeNPs. However, when this co-transfection exists in FeNPs, it has a very significant killing effect on cancer cells, and its effect The killing effect of each vector alone in combination with FeNPs on cancer cells was exceeded (FIG. 38-FIG. 40). It shows that the five genes have synergistic effect. The viability of various cells under various treatments was determined by the CCK8 method, and the results were consistent with the results of acridine orange/ethidium bromide staining, and more clearly showed that the five genes had significant synergistic effects (FIG. 43).

What is claimed is:

1. A composition for killing cancer cells, comprising
a gene interference vector and iron nanoparticles,
wherein
the gene interference vector is a CRISPR/Cas13a expression vector or a microRNA expression vector controlled by a cancer cell specific promoter DMP;
the Cas13a-gRNA expressed by the CRISPR/Cas13a expression vector or the microRNA expressed by the microRNA expression vector is configured to inhibit, in a targeted manner, intracellular iron metabolism and expression of reactive oxygen species-related genes; and
the iron nanoparticles are iron nanomaterials that are configured to be degraded after entering cells to generate iron ions and to increase the level of intracellular reactive oxygen species levels.

2. (canceled)

3. The composition for killing cancer cells according to claim 1, wherein
the cancer cell specific promoter DMP is a NF-κB-specific promoter, which is composed of NF-κB;
the κB decoy and a minimal promoter are connected to form, the promoter is configured to activate its downstream genes to be expressed in various cancer cells, but not in normal cells; the DMP promoter can control the expression of CRISPR/Cas13a or microRNA expression vector in cancer cells specific expression in.

4. The composition for killing cancer cells according to claim 1, wherein
in the CRISPR/Cas13a expression vector,
the expression of Cas13a is controlled by the DMP promoter, and
the expression of gRNA is controlled by the U6 promoter;
the expression of the microRNA is controlled by the DMP promoter in the microRNA expression vector.

5. The composition for killing cancer cells according to claim 1, wherein
the DNA sequence of the functional element of the CRISPR/Cas13a expression vector is shown in SEQ ID NO.1;
the DNA sequence of the functional element of the microRNA expression vector is shown in SEQ ID NO.2.

6. The composition for killing cancer cells according to claim 1, wherein
the CRISPR/Cas13a or microRNA expression vector is configured to
express either a single gene-targeting gRNA or microRNA; or
as a combined CRISPR/Cas13a or microRNA expression vector to express gRNA or microRNA targeting multiple genes.

7. The composition for killing cancer cells according to claim 2, wherein the iron metabolism and reactive oxygen species-related genes is selected from FPN, LCN2, FSP1, FTH1, GPX4, NRF2 and SLC7A11 genes.

8. The composition for killing cancer cells according to claim 7, wherein the CRISPR/Cas13a or microRNA expression vector is configured to express gRNA or microRNA targeting a group of genes selected from FPN, LCN2, FSP1, FTH1, GPX4, NRF2 and SLC7A11 genes, in which gRNA can form a first complex with Cas13a protein, and microRNA can form a second complex with RISC, and both first and second complexes can target cleavage of the mRNA of the group of genes, causing a reduction of expression level of proteins encoded by the group of genes.

9. The composition for killing cancer cells according to claim 7, wherein
target binding sequences of the gRNAs targeting FPN and LCN2 are:
(FPN)
5′-CACCG CAAAG TGCCA CATCC GATCT CCC-3′
and
(LCN2)
5′-TAACT CTTAA TGTTG CCCAG CGTGA ACT-3′;
target binding sequences of the microRNAs targeting FPN, LCN2, FSP1, FTH1, GPX4, NRF2 and SLC7A11 genes are:
(FPN)
5′-TCTAC CTGCA GCTTA CATGA T-3′,
(LCN2)
5′-TAATG TTGCC CAGCG TGAAC T-3′,
(FSP1)
5′-CAAAC AAACA AATAA AGTGG A-3′,
(FSP1)
5′-TAAAC AAACA AACAA ATAAA G-3′,
(FTH1)
5′-ATCCC AAGAC CTCAA AGACA A-3′,
(FTH1)
5′-TAAGG AATCT GGAAG ATAGC C-3′,
(GPX4)
5′-TTCAG TAGGC GGCAA AGGCG G-3′,
(GPX4)
5′-AGGAA CTGTG GAGAG ACGGT G-3′,
(NRF2)
5′-TACTG ATTCA ACATA CTGAC A-3′,
(NRF2)
5′-TTTAC ACTTA CACAG AAACT A-3′,
(SLC7A11)
5′-AAATG ATACA GCCTT AACAC A-3′
and
(SLC7A11)
5′-TTGAG TTGAG GACCA GTTAG T-3′.

10. The composition for killing cancer cells according to claim 1, wherein the iron nanoparticles are FeNPs or FeNCs.

11. The composition for killing cancer cells according to claim 1, wherein
under the combined action of gene interference vector and iron nanoparticles, the levels of iron ions and reactive oxygen species in cancer cells can be sharply increased, inducing significant ferroptosis in cancer cells.

12. The composition for killing cancer cells according to claim 1, wherein the gene interference vector is configured to be administered in vivo in a form of viral vectors including adeno-associated virus and other non-viral vectors including nanocarriers; and
the iron nanoparticles is configured to be administered in vivo either as a separate chemical material or as a nanocarrier of gene interference vector for in vivo administration concurrently.

13. The composition for killing cancer cells according to claim 1, wherein the composition for killing cancer cells of claim 1 is used in a preparation of novel cancer therapeutic agents.


SEQUENCE LISTING (2 sequences)

SEQ ID NO: 1 - DNA, 4246 bp, Artificial Sequence
  Synthetic gene
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatttagactaccccaaaaacgaaggggactaaaacgggtcttcgagaagacctggatccggcctaactggccggtaccgggaatttccggggactttccgggaatttccggggactttccgggaatttcctagagggtatataatggaagctcgacttccaggtcgacgcgtaagcttgccaccatgccaaagaagaagcggaaggtcggtatccacggagtcccagcagccatgaaagtgaccaaggtcgacggcatcagccacaagaagtacatcgaagagggcaagctcgtgaagtccaccagcgaggaaaaccggaccagcgagagactgagcgagctgctgagcatccggctggacatctacatcaagaaccccgacaacgcctccgaggaagagaaccggatcagaagagagaacctgaagaagttctttagcaacaaggtgctgcacctgaaggacagcgtgctgtatctgaagaaccggaaagaaaagaacgccgtgcaggacaagaactatagcgaagaggacatcagcgagtacgacctgaaaaacaagaacagcttctccgtgctgaagaagatcctgctgaacgaggacgtgaactctgaggaactggaaatctttcggaaggacgtggaagccaagctgaacaagatcaacagcctgaagtacagcttcgaagagaacaaggccaactaccagaagatcaacgagaacaacgtggaaaaagtgggcggcaagagcaagcggaacatcatctacgactactacagagagagcgccaagcgcaacgactacatcaacaacgtgcaggaagccttcgacaagctgtataagaaagaggatatcgagaaactgtttttcctgatcgagaacagcaagaagcacgagaagtacaagatccgcgagtactatcacaagatcatcggccggaagaacgacaaagagaacttcgccaagattatctacgaagagatccagaacgtgaacaacatcaaagagctgattgagaagatccccgacatgtctgagctgaagaaaagccaggtgttctacaagtactacctggacaaagaggaactgaacgacaagaatattaagtacgccttctgccacttcgtggaaatcgagatgtcccagctgctgaaaaactacgtgtacaagcggctgagcaacatcagcaacgataagatcaagcggatcttcgagtaccagaatctgaaaaagctgatcgaaaacaaactgctgaacaagctggacacctacgtgcggaactgcggcaagtacaactactatctgcaagtgggcgagatcgccacctccgactttatcgcccggaaccggcagaacgaggccttcctgagaaacatcatcggcgtgtccagcgtggcctacttcagcctgaggaacatcctggaaaccgagaacgagaacggtatcaccggccggatgcggggcaagaccgtgaagaacaacaagggcgaagagaaatacgtgtccggcgaggtggacaagatctacaatgagaacaagcagaacgaagtgaaagaaaatctgaagatgttctacagctacgacttcaacatggacaacaagaacgagatcgaggacttcttcgccaacatcgacgaggccatcagcagcatcagacacggcatcgtgcacttcaacctggaactggaaggcaaggacatcttcgccttcaagaatatcgcccccagcgagatctccaagaagatgtttcagaacgaaatcaacgaaaagaagctgaagctgaaaatcttcaagcagctgaacagcgccaacgtgttcaactactacgagaaggatgtgatcatcaagtacctgaagaataccaagttcaacttcgtgaacaaaaacatccccttcgtgcccagcttcaccaagctgtacaacaagattgaggacctgcggaataccctgaagtttttttggagcgtgcccaaggacaaagaagagaaggacgcccagatctacctgctgaagaatatctactacggcgagttcctgaacaagttcgtgaaaaactccaaggtgttctttaagatcaccaatgaagtgatcaagattaacaagcagcggaaccagaaaaccggccactacaagtatcagaagttcgagaacatcgagaaaaccgtgcccgtggaatacctggccatcatccagagcagagagatgatcaacaaccaggacaaagaggaaaagaatacctacatcgactttattcagcagattttcctgaagggcttcatcgactacctgaacaagaacaatctgaagtatatcgagagcaacaacaacaatgacaacaacgacatcttctccaagatcaagatcaaaaaggataacaaagagaagtacgacaagatcctgaagaactatgagaagcacaatcggaacaaagaaatccctcacgagatcaatgagttcgtgcgcgagatcaagctggggaagattctgaagtacaccgagaatctgaacatgttttacctgatcctgaagctgctgaaccacaaagagctgaccaacctgaagggcagcctggaaaagtaccagtccgccaacaaagaagaaaccttcagcgacgagttggaactgatcaacctgctgaacctggacaacaacagagtgaccgaggacttcgagctggaagccaacgagatcggcaagttcctggacttcaacgaaaacaaaatcaaggaccggaaagagctgaaaaagttcgacaccaacaagatctatttcgacggcgagaacatcatcaagcaccgggccttctacaatatcaagaaatacggcatgctgaatctgctggaaaagatcgccgataaggccaagtataagatcagcctgaaagaactgaaagagtacagcaacaagaagaatgagattgaaaagaactacaccatgcagcagaacctgcaccggaagtacgccagacccaagaaggacgaaaagttcaacgacgaggactacaaagagtatgagaaggccatcggcaacatccagaagtacacccacctgaagaacaaggtggaattcaatgagctgaacctgctgcagggcctgctgctgaagatcctgcaccggctcgtgggctacaccagcatctgggagcgggacctgagattccggctgaagggcgagtttcccgagaaccactacatcgaggaaattttcaatttcgacaactccaagaatgtgaagtacaaaagcggccagatcgtggaaaagtatatcaacttctacaaagaactgtacaaggacaatgtggaaaagcggagcatctactccgacaagaaagtgaagaaactgaagcaggaaaaaaaggacctgtacatccggaactacattgcccacttcaactacatcccccacgccgagattagcctgctggaagtgctggaaaacctgcggaagctgctgtcctacgaccggaagctgaagaacgccatcatgaagtccatcgtggacattctgaaagaatacggcttcgtggccaccttcaagatcggcgctgacaagaagatcgaaatccagaccctggaatcagagaagatcgtgcacctgaagaatctgaagaaaaagaaactgatgaccgaccggaacagcgaggaactgtgcgaactcgtgaaagtcatgttcgagtacaaggccctggaaaaaaggccggcggccacgaaaaaggccggccaggcaaaaaagaaaaagtgactcgagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatctta

SEQ ID NO: 2 - DNA, 860 bp, Artificial Sequence
  Synthetic gene
gggaatttccggggactttccgggaatttccggggactttccgggaatttcctagagggtatataatggaagctcgacttccaggctagcgaattcgctaagcacttcgtggccgtcgatcgtttaaagggaggtagtgagtcgaccagtggatcctggaggcttgctgaaggctgtatgctggagacgcagtgagccgagatcgcgccaccgcgtctcgcaggacacaaggcctgttactagcactcacatggaacaaatggcccagatctggccgcactcgagatatctagacccagctttcttgtacaaagtggttgatctagagggcccgcggttcgctgatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgtgaccccattggggccaatacgcccgcgtttcttccttttccccaccccaccccccaagttcgggtgaaggcccagggctcgcagccaacgtcggggcggcaggccctgccataggcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta